(19) United States
(12) Patent Application Publication
(10) Pub. No.: US 2008/0161306 A1
(43) Pub. Date: Jul. 3, 2008

(54) Pyrrole Derivatives as Dna Gyrase and Topoisomerase Inhibitors

(76) Inventors: Brian Sherer, Nashua, NH (US); Fei Zhou, Southborough, MA (US)

Correspondence Address: ASTRAZENECA R&D BOSTON, 35 GATEHOUSE DRIVE, WALTHAM, MA, 02451-1215, US

(21) Appl. No.: 11/816,450

(22) PCT Filed: Feb. 16, 2006
(86) PCT No.: PCT/GB06/00537

§ 371 Date: Aug. 16, 2007

Related U.S. Application Data
(60) Provisional application No. 60/654,385, filed on Feb. 18, 2005.

(51) Int. Cl. A61K 31/5377; C07D 207/36; A61K 31/40; C07D 413/02

(52) U.S. Cl. 514/235.5; 548/537; 514/423; 544/141

(57) ABSTRACT

Compounds of formula (I) and their pharmaceutically acceptable salts are described: Processes for their preparation, pharmaceutical compositions containing them, their use as medicaments and their use in the treatment of bacterial infections are also described.

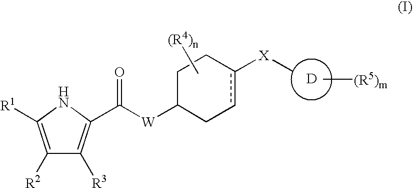

### BACKGROUND OF THE INVENTION

The present invention relates to compounds which demonstrate antibacterial activity, processes for their preparation, pharmaceutical compositions containing them as the active ingredient, to their use as medicaments and to their use in the manufacture of medicaments for use in the treatment of bacterial infections in warm-blooded animals such as humans. In particular this invention relates to compounds useful for the treatment of bacterial infections in warm-blooded animals such as humans, more particularly to the use of these compounds in the manufacture of medicaments for use in the treatment of bacterial infections in warm-blooded animals such as humans.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus aureus (MRSA), methicillin resistant coagulase negative staphylococci (MRCNS), penicillin resistant Streptococcus pneumoniae and multiple resistant Enterococcus faecium.

The preferred clinically effective antibiotic for treatment of last resort of such resistant Gram-positive pathogens is vancomycin. Vancomycin is a glycopeptide and is associated with various toxicities, including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Consequently, in order to overcome the threat of widespread multi-drug resistant organisms, there is an on-going need to develop new antibiotics, particularly those with either a novel mechanism of action and/or containing new pharmacophoric groups.

Deoxyribonucleic acid (DNA) gyrase is a member of the type II family of topoisomerases that control the topological state of DNA in cells (Champoux, J. J.; 2001. Ann. Rev. Biochem. 70: 369-413). Type II topoisomerases use the free energy from adenosine triphosphate (ATP) hydrolysis to alter the topology of DNA by introducing transient double-stranded breaks in the DNA, catalyzing strand passage through the break and resealing the DNA. DNA gyrase is an essential and conserved enzyme in bacteria and is unique among topoisomerases in its ability to introduce negative supercoils into DNA. The enzyme consists of two subunits, encoded by gyrA and gyrB, forming an A₂B₂ tetrameric complex. The A subunit of gyrase (GyrA) is involved in DNA breakage and resealing and contains a conserved tyrosine residue that forms the transient covalent link to DNA during strand passage. The B subunit (GyrB) catalyzes the hydrolysis of ATP and interacts with the A subunit to translate the free energy from hydrolysis to the conformational change in the enzyme that enables strand-passage and DNA resealing.

Another conserved and essential type II topoisomerase in bacteria, called topoisomerase IV, is primarily responsible for separating the linked closed circular bacterial chromosomes produced in replication. This enzyme is closely related to DNA gyrase and has a similar tetrameric structure formed from subunits homologous to Gyr A and to Gyr B. The overall sequence identity between gyrase and topoisomerase IV in different bacterial species is high. Therefore, compounds that target bacterial type II topoisomerases have the potential to inhibit two targets in cells, DNA gyrase and topoisomerase IV; as is the case for existing quinolone antibacterials (Maxwell, A. 1997, Trends Microbiol. 5: 102-109).

DNA gyrase is a well-validated target of antibacterials, including the quinolones and the coumarins. The quinolones (e.g. ciprofloxacin) are broad-spectrum antibacterials that inhibit the DNA breakage and reunion activity of the enzyme and trap the GyrA subunit covalently complexed with DNA (Drlica, K., and X. Zhao, 1997, Microbiol. Molec. Biol. Rev. 61: 377-392). Members of this class of antibacterials also inhibit topoisomerase IV and as a result, the primary target of these compounds varies among species. Although the quinolones are successful antibacterials, resistance generated primarily by mutations in the target (DNA gyrase and topoisomerase IV) is becoming an increasing problem in several organisms, including S. aureus and Streptococcus pneumoniae (Hooper, D. C., 2002, The Lancet Infectious Diseases 2: 530-538). In addition, quinolones, as a chemical class, suffer from toxic side effects, including arthropathy that prevents their use in children (Lipsky, B. A. and Baker, C. A., 1999, Clin. Infect. Dis. 28: 352-364). Furthermore, the potential for cardiotoxicity, as predicted by prolongation of the QT_{c} interval, has been cited as a toxicity concern for quinolones.

There are several known natural product inhibitors of DNA gyrase that compete with ATP for binding the GyrB subunit (Maxwell, A. and Lawson, D. M. 2003, Curr. Topics in Med. Chem. 3: 283-303). The coumarins are natural products isolated from Streptomyces spp., examples of which are novobiocin, chlorobiocin and coumermycin A1. Although these compounds are potent inhibitors of DNA gyrase, their therapeutic utility is limited due to toxicity in eukaryotes and poor penetration in Gram-negative bacteria (Maxwell, A. 1997, Trends Microbiol. 5: 102-109). Another natural product class of compounds that targets the GyrB subunit is the cyclothialidines, which are isolated from Streptomyces filipensis (Watanabe, J. et al 1994, J. Antibiot. 47: 32-36). Despite potent activity against DNA gyrase, cyclothialidine is a poor antibacterial agent showing activity only against some eubacterial species (Nakada, N, 1993, Antimicrob. Agents Chemother. 37: 2656-2661).

Synthetic inhibitors that target the B subunit of DNA gyrase and topoisomeraseIV are known in the art. For example, coumarin-containing compounds are described in patent application number WO 99/35155, 5,6-bicyclic heteroaromatic compounds are described in patent application WO 02/060879, and pyrazole compounds are described in patent application WO 01/52845 (U.S. Pat. No. 6,608,087).

We have discovered a new class of compounds which are useful for inhibiting DNA gyrase and topoisomerase IV.

### SUMMARY OF THE INVENTION

Therefore the present invention provides a compound of formula (I):

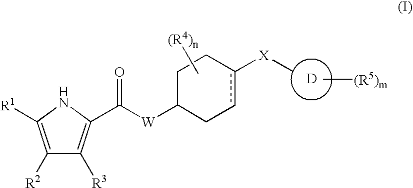

wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;

R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;

W is —O—, —N(R⁶)— or —C(R⁷)(R⁸)—;

“—” is a bond or is absent;

X is a direct bond, —CH₂—, —C(O)— or S(O)_{q}— (wherein q is 1 or 2);

Ring D is carbocyclyl or heterocyclyl; wherein if said heterocyclyl contains an —NH— moiety that nitrogen may be optionally substituted by a group selected from R⁹; R⁴ and R⁵ are substituents on carbon and are independently selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁰— or heterocyclyl-R¹¹—; wherein R⁴ and R⁵ independently of each other may be optionally substituted on carbon by one or more R¹²; and wherein if said heterocyclyl contains an —NH— moiety that nitrogen may be optionally substituted by a group selected from R¹³;

R⁶, R⁷ and R⁸ are independently selected from hydrogen or C₁₋₄alkyl;

n is 0-4; wherein the values of R⁴ may be the same or different;

m is 0-4; wherein the values of R⁵ may be the same or different;

R¹² is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N-(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹⁴— or heterocyclyl-R¹⁵—; wherein R¹² independently of each other may be optionally substituted on carbon by one or more R¹⁶; and wherein if said heterocyclyl contains an —NH— moiety that nitrogen may be optionally substituted by a group selected from R¹⁷;

R⁹, R¹³ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁰, R¹¹, R¹⁴ and R¹⁵ are independently selected from a direct bond, —O—, —N(R¹⁸)—, —C(O)—, —N(R¹⁹)C(O)—, —C(O)N(R²⁰)—, —S(O)ₚ—, —SO₂N(R²¹)— or —N(R²²)SO₂—; wherein R¹⁸, R¹⁹, R²⁰, R²¹ and R²² are independently selected from hydrogen or C₁₋₄alkyl and p is 0-2;

R¹⁶ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

What is also provided is a compound of formula IA:

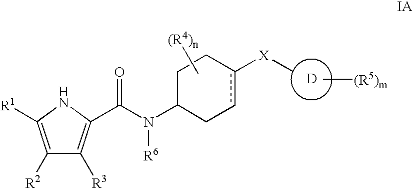

wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;

R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;

“—” is a bond or is absent;

X is a direct bond, —CH₂—, —C(O)— or S(O)_{q}— (wherein q is 1 or 2);

Ring D is carbocyclyl or heterocyclyl; wherein if said heterocyclyl contains an —NH— moiety that nitrogen may be optionally substituted by a group selected from R⁹;

R⁴ and R⁵ are substituents on carbon and are independently selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N-(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′-(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N-(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁰— or heterocyclyl-R¹¹—; wherein R⁴ and R⁵ independently of each other may be optionally substituted on carbon by one or more R¹²; and wherein if said heterocyclyl contains an —NH— moiety that nitrogen may be optionally substituted by a group selected from R¹³;

R⁶, R⁷ and R⁸ are independently selected from hydrogen or C₁₋₄alkyl;

n is 0-4; wherein the values of R⁴ may be the same or different;

m is 0-4; wherein the values of R⁵ may be the same or different;

R¹² is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹⁴— or heterocyclyl-R¹⁵—; wherein R¹² independently of each other may be optionally substituted on carbon by one or more R¹⁶; and wherein if said heterocyclyl contains an —NH— moiety that nitrogen may be optionally substituted by a group selected from R¹⁷;

R⁹, R¹³ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁰, R¹¹, R¹⁴ and R¹⁵ are independently selected from a direct bond, —O—, —N(R¹⁸)—, —C(O)—, —N(R¹⁹)C(O)—, —C(O)N(R²⁰)—, —S(O)ₚ—, —SO₂N(R²¹)— or —N(R²²)SO₂—; wherein R¹⁸, R¹⁹, R²⁰, R²¹ and R²² are independently selected from hydrogen or C₁₋₄alkyl and p is 0-2;

R¹⁶ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

What is also provided is a compound of formula IB:

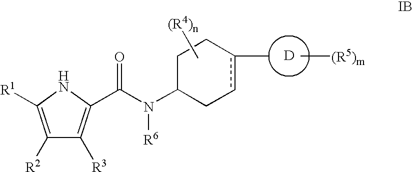

wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;

R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;

“—” is a bond or is absent;

Ring D is carbocyclyl or heterocyclyl; wherein if said heterocyclyl contains an —NH— moiety that nitrogen may be optionally substituted by a group selected from R⁹;

R⁴ and R⁵ are substituents on carbon and are independently selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N′(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁰— or heterocyclyl-R¹¹—; wherein R⁴ and R⁵ independently of each other may be optionally substituted on carbon by one or more R¹²; and wherein if said heterocyclyl contains an —NH— moiety that nitrogen may be optionally substituted by a group selected from R¹³;

R⁶, R⁷ and R⁸ are independently selected from hydrogen or C₁₋₄alkyl;

n is 0-4; wherein the values of R⁴ may be the same or different;

m is 0-4; wherein the values of R⁵ may be the same or different;

R¹² is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹⁴— or heterocyclyl-R¹⁵—; wherein R¹² independently of each other may be optionally substituted on carbon by one or more R¹⁶; and wherein if said heterocyclyl contains an —NH— moiety that nitrogen may be optionally substituted by a group selected from R¹⁷;

R⁹, R¹³ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁰, R¹¹, R¹⁴ and R¹⁵ are independently selected from a direct bond, —O—, —N(R¹⁸)—, —C(O)—, —N(R¹⁹)C(O)—, —C(O)N(R²⁰)—, —S(O)ₚ—, —SO₂N(R²¹)— or —N(R²²)SO₂—; wherein R¹⁸, R¹⁹, R²⁰, R²¹ and R²² are independently selected from hydrogen or C₁₋₄alkyl and p is 0-2;

R¹⁶ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

What is also provided is a compound of formula IC:

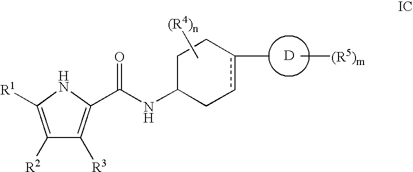

wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;

R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₅cycloalkyl; wherein R³ may be optionally substituted on carbon by one or more halo or C₃₋₆cycloalkyl;

“—” is a bond or is absent;

Ring D is heterocyclyl; wherein if said heterocyclyl contains an —NH— moiety that nitrogen may be optionally substituted by a group selected from R⁹;

R⁴ and R⁵ are substituents on carbon and are independently selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁰— or heterocyclyl-R¹¹—; wherein R⁴ and R⁵ independently of each other may be optionally substituted on carbon by one or more R¹²; and wherein if said heterocyclyl contains an —NH— moiety that nitrogen may be optionally substituted by a group selected from R¹³;

R⁶, R⁷ and R⁸ are independently selected from hydrogen or C₁₋₄alkyl;

n is 0-4; wherein the values of R⁴ may be the same or different;

m is 0-4; wherein the values of R⁵ may be the same or different;

R¹² is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹⁴— or heterocyclyl-R¹⁵—; wherein R¹² independently of each other may be optionally substituted on carbon by one or more R¹⁶; and wherein if said heterocyclyl contains an —NH— moiety that nitrogen may be optionally substituted by a group selected from R¹⁷;

R⁹, R¹³ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁰, R¹¹, R¹⁴ and R¹⁵ are independently selected from a direct bond, —O—, —N(R¹⁸)—, —C(O)—, —N(R¹⁹)C(O)—, —C(O)N(R²⁰)—, —S(O)ₚ—, —SO₂N(R²¹)— or —N(R²²)SO₂—; wherein R¹⁸, R¹⁹, R²⁰, R²¹ and R²² are independently selected from hydrogen or C₁₋₄alkyl and p is 0-2;

R¹⁶ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

What is also provided is a compound of formula ID:

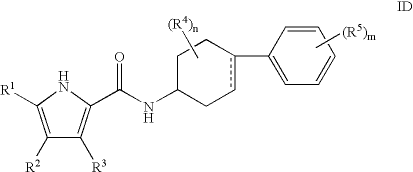

wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;

R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;

“—” is a bond or is absent;

R⁴ and R⁵ are substituents on carbon and are independently selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)aamino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁰— or heterocyclyl-R¹¹—; wherein R⁴ and R⁵ independently of each other may be optionally substituted on carbon by one or more R¹²; and wherein if said heterocyclyl contains an —NH— moiety that nitrogen may be optionally substituted by a group selected from R¹³;

R⁶, R⁷ and R⁸ are independently selected from hydrogen or C₁₋₄alkyl;

n is 0-4; wherein the values of R⁴ may be the same or different;

m is 0-4; wherein the values of R⁵ may be the same or different;

R¹² is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹⁴— or heterocyclyl-R¹⁵—; wherein R¹² independently of each other may be optionally substituted on carbon by one or more R¹⁶; and wherein if said heterocyclyl contains an —NH— moiety that nitrogen may be optionally substituted by a group selected from R¹⁷;

R⁹, R¹³ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁰, R¹¹, R¹⁴ and R¹⁵ are independently selected from a direct bond, —O—, —N(R¹⁸)—, —C(O)—, —N(R¹⁹)C(O)—, —C(O)N(R²⁰)—, —S(O)ₚ—, —SO₂N(R²¹)— or —N(R²²)SO₂—; wherein R¹⁸, R¹⁹, R²⁰, R²¹ and R²² are independently selected from hydrogen or C₁₋₄alkyl and p is 0-2;

R¹⁶ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

What is also provided is a compound of formula IE:

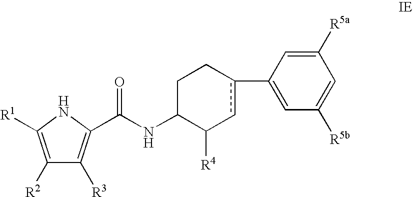

wherein:

R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;

R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;

R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;

“—” is a bond or is absent;

R⁴, R^{5a}, and R^{5b} are substituents on carbon and are independently selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁰— or heterocyclyl-R¹¹—; wherein R⁴ and R⁵ independently of each other may be optionally substituted on carbon by one or more R¹²; and wherein if said heterocyclyl contains an —NH— moiety that nitrogen may be optionally substituted by a group selected from R¹³;

R⁶, R⁷ and R⁸ are independently selected from hydrogen or C₁₋₄alkyl;

n is 0-4; wherein the values of R⁴ may be the same or different;

m is 0-4; wherein the values of R⁵ may be the same or different;

R¹² is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹⁴— or heterocyclyl-R¹⁵—; wherein R¹² independently of each other may be optionally substituted on carbon by one or more R¹⁶; and wherein if said heterocyclyl contains an —NH— moiety that nitrogen may be optionally substituted by a group selected from R¹⁷;

R⁹, R¹³ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁰, R¹¹, R¹⁴ and R¹⁵ are independently selected from a direct bond, —O—, —N(R¹⁸)—, —C(O)—, —N(R¹⁹)C(O)—, —C(O)N(R²⁰)—, —S(O)ₚ—, —SO₂N(R²¹)— or —N(R²²)SO₂—; wherein R¹⁸, R¹⁹, R²⁰, R²¹ and R²² are independently selected from hydrogen or C₁₋₄alkyl and p is 0-2;

R¹⁶ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

The invention also provides a compound which is

trans-Ethyl 3-(4-{[(3,4-dichloro-5-methyl-1H-pyrrol-2-yl)carbonyl]amino}cyclohexyl)benzoate;

cis-Ethyl 3-(4-{[(3,4-dichloro-5-methyl-1H-pyrrol-2-yl)carbonyl]amino}cyclohexyl)benzoate;

Ethyl 3-(4-{[(3,4-dichloro-5-methyl-1H-pyrrol-2-yl)carbonyl]amino}-1-hydroxycyclohexyl)benzoate;

Dimethyl 5-(4-{[(3,4-dichloro-5-methyl-1H-pyrrol-2-yl)carbonyl]amino}cyclohex-1-en-1-yl)isophthalate;

trans-3-(4-{[(3,4-Dichloro-5-methyl-1H-pyrrol-2-yl)carbonyl]amino}cyclohexyl)benzoic acid;

cis-3-(4-{[(3,4-Dichloro-5-methyl-1H-pyrrol-2-yl)carbonyl]amino}cyclohexyl)benzoic acid;

3-(4-{[(3,4-Dichloro-5-methyl-1H-pyrrol-2-yl)carbonyl]amino}-1-hydroxycyclohexyl)benzoic acid;

5-(4-{[(3,4-Dichloro-5-methyl-1H-pyrrol-2-yl)carbonyl]amino}cyclohex-1-en-yl)isophthalic acid;

3-(trans-4-{[(3,4-Dichloro-5-methyl-1H-pyrrol-2-yl)carbonyl]amino}-3-methoxy cyclohex-1-en-1-yl)benzoic acid;

3,4-Dichloro-N-(4-1{3-[(methoxyamino)carbonyl]phenyl}cyclohex-3-en-1-yl)-5-methyl-1H-pyrrole-2-carboxamide;

3-((3,4-Cis)-4-{[(3,4-Dichloro-5-methyl-1H-pyrrol-2-yl)carbonyl]amino}-3-methoxycyclohex-1-en-1-yl)benzoic acid;

Ethyl 3-(trans-4-{[(3,4-dichloro-5-methyl-1H-pyrrol-2-yl)carbonyl]amino}-3-methoxy cyclohex-1-en-1-yl)benzoate;

3,4-Dichloro-N-(trans-2-methoxy-4-{3-[(methylsulfonyl)amino]phenyl}cyclohex-3-en-1-yl)-5-methyl-1H-pyrrole-2-carboxamide;

3,4-Dichloro-N-{trans-2-methoxy-4-[3-(morpholin-4-ylcarbonyl)phenyl]cyclohex-3-en-1-yl}-5-methyl-1H-pyrrole-2-carboxamide;

3,4-Dichloro-N-{trans-2-methoxy-4-[3-(methylsulfonyl)phenyl]cyclohex-3-en-1-yl}-5-methyl-1H-pyrrole-2-carboxamide;

5-(4-{[(3,4-Dichloro-5-methyl-1H-pyrrol-2-yl)carbonyl]amino}cyclohex-1-en-1-yl)benzoic acid;

or a pharmaceutically acceptable salt thereof.

The invention also provides a pharmaceutical composition that comprises a compound of formula I, IA, IB, IC, ID, or IE, or a pharmaceutically-acceptable salt thereof, and a pharmaceutically-acceptable diluent or carrier.

The invention also provides a method of treating a bacterial infection in a warm-blooded animal, such as a human being, in need of such treatment, which comprises administering to said animal an effective amount of a compound of formula I, IA, IB, IC, ID, or IE, or a pharmaceutically-acceptable salt thereof.

The invention also provides a method for inhibiting bacterial DNA gyrase in a warm-blooded animal, such as a human being, in need of such treatment which comprises administering to said animal an effective amount of a compound of formula I, IA, IB, IC, ID, or IE, or a pharmaceutically acceptable salt.

The invention also provides a compound of formula I, IA, IB, IC, ID, or IE, and pharmaceutically acceptable salts thereof for use as a medicament.

The invention also provides the use of a compound of formula I, IA, IB, IC, ID, or IE, or a pharmaceutically acceptable salt thereof in the manufacture of a medicament for use in the production of an anti-bacterial effect in a warm-blooded animal such as a human being.

The invention also provides the use of a compound of formula I, IA, IB, IC, ID, or IE, a pharmaceutically acceptable salt thereof in the manufacture of a medicament for use in the treatment of a bacterial infection in a warm-blooded animal such as a human being.

What is also provided is a process for preparing a compound of formula I, IA, IB, IC, ID, or IE, or a pharmaceutically-acceptable salt thereof, comprising:
Process a) for compounds of formula (I) wherein W is —C(R⁷)(R⁸)—; converting a compound of formula (II):

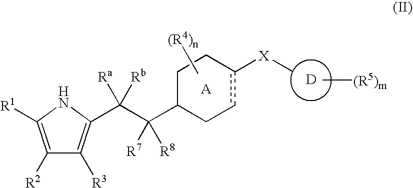

wherein R^{a} is cyano and R^{b} is dimethyamino or diethylamino; or R^{a} and R^{b} are independently selected from C₁₋₄alkylthio; or R^{a} and R^{b} together form 1,3-dithianyl or 1,3-dithiolanyl; into a compound of formula (I);
Process b) for compounds of formula (I) wherein W is —O—; reacting a compound of formula (III):

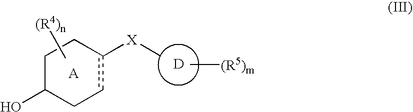

with a compound of formula (IV):

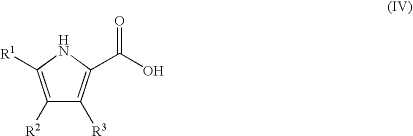

Process c) for compounds of formula (I) wherein W is —N(R⁶)—; reacting a compound of formula (V):

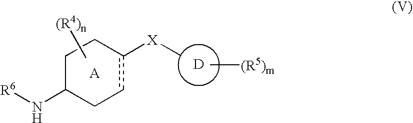

with a compound of formula (IV) or an activated acid derivative thereof;
Process d) for compounds of formula (I) wherein W is —C(R⁷)(R⁸)—; reacting a compound of formula (VI):

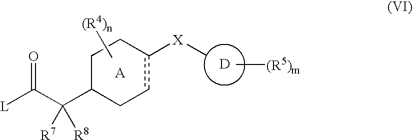

wherein L is a displaceable group; with a compound of formula (VII):

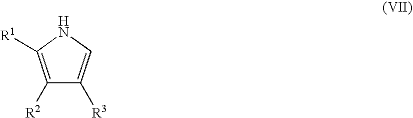

Process e) for compounds of formula (I) wherein W is —C(R⁷)(R⁸)—; reacting a compound of formula (VIII):

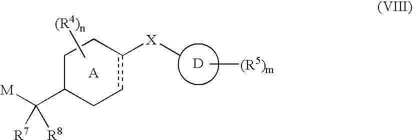

wherein M is an organometallic group; with a compound of formula (IX):

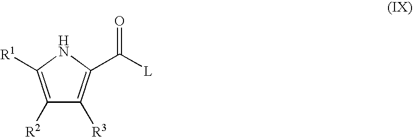

wherein L is a displaceable group;
Process f) for compounds of formula (I) wherein Ring A is cyclohexenyl; reacting a compound of formula (X):

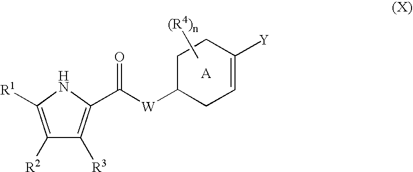

with a compound of formula (XI):

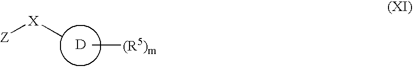

wherein one of Y and Z is an organometallic group and the other is a displaceable group;
Process g) for compounds of formula (I) wherein Ring A is cyclohexyl, by reducing a compound of formula (I) wherein Ring A is cyclohexenyl;
Process h) for compounds of formula (I) wherein Ring A is cyclohexenyl, by dehydrating a compound of formula (XII);

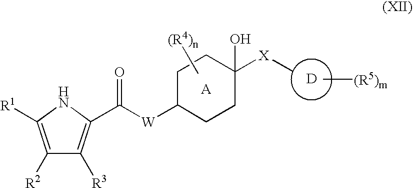

and thereafter if necessary:
i) converting a compound of the formula (I) into another compound of the formula (I);
ii) removing any protecting groups;
iii) forming a pharmaceutically acceptable salt.

### DETAILED DESCRIPTION OF THE INVENTION

In this specification the term alkyl includes both straight and branched chain alkyl groups. For example, “C₁₋₄alkyl” includes methyl, ethyl, propyl, isopropyl and t-butyl. However references to individual alkyl groups such as propyl are specific for the straight chain version only. An analogous convention applies to other generic terms.

Where optional substituents are chosen from one or more groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring nitrogen and/or a ring sulphur atom may be optionally oxidized to form the N— or S-oxide(s). In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidized to form the S-oxides. In a further aspect of the invention a “heterocyclyl” is an unsaturated, carbon-linked, monocyclic ring containing 5 or 6 atoms of which at least one atom is chosen from nitrogen, sulphur or oxygen. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl.

An example of “C₁₋₄alkanoyloxy” is acetoxy. Examples of “C₁₋₄alkoxycarbonyl” include methoxycarbonyl, ethoxycarbonyl, n- and t-butoxycarbonyl. Examples of “C₁₋₄alkoxycarbonylamino” include methoxycarbonylamino, ethoxycarbonylamino, n- and t-butoxycarbonylamino. Examples of “C₁₋₄alkoxy” include methoxy, ethoxy and propoxy. Examples of “C₁₋₄alkanoylamino” include formamido, acetamido and propionylamino. Examples of “C₁₋₄alkylS(O)ₐ wherein a is 0 to 2” include methylthio, ethylthio, methylsulphinyl, ethylsulphinyl, mesyl and ethylsulphonyl. Examples of “C₁₋₄alkanoyl” include propionyl and acetyl. Examples of “N—(C₁₋₄alkyl)amino” include methylamino and ethylamino. Examples of “N,N—(C₁₋₄alkyl)₂amino” include di-N-methylamino, di-(N-ethyl)amino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl. Examples of “N—(C₁₋₄alkyl)sulphamoyl” are N-(methyl)sulphamoyl and N-(ethyl)sulphamoyl. Examples of “N,N—(C₁₋₄alkyl)₂sulphamoyl” are N,N-(dimethyl)sulphamoyl and N-(methyl)-N-(ethyl)sulphamoyl. Examples of “N—(C₁₋₄alkyl)carbamoyl” are methylaminocarbonyl and ethylaminocarbonyl. Examples of “N,N—(C₁₋₄alkyl)₂carbamoyl” are dimethylaminocarbonyl and methylethylaminocarbonyl. Examples of “N—(C₁₋₄alkoxy)carbamoyl” are methoxyaminocarbonyl and isopropoxyaminocarbonyl. Examples of “N—(C₁₋₄alkyl)-N—(C₁₋₄alkoxy)carbamoyl” are N-methyl-N-methoxyaminocarbonyl and N-methyl-N-ethoxyaminocarbonyl. Examples of “C₃₋₆cycloalkyl” are cyclopropyl, cyclobutyl, cyclopropyl and cyclohexyl. Examples of “N′—(C₁₋₄alkyl)ureido” are N′-methylureido and N′-isopropylureido. Examples of “N′,N′—(C₁₋₄alkyl)₂ureido” are N′N′-dimethylureido and N′-methyl-N′-isopropylureido. Examples of “N′—(C₁₋₄alkyl)hydrazinocarbonyl” are N′-methylhydrazinocarbonyl and N′-isopropylhydrazinocarbonyl. Examples of “N′,N′—(C₁₋₄alkyl)₂hydrazinocarbonyl” are N′N′-dimethylhydrazinocarbonyl and N′-methyl-N′-isopropylhydrazinocarbonyl. Examples of “C₁₋₄alkylsulphonylamino” include methylsulphonylamino, isopropylsulphonylamino and t-butylsulphonylamino. Examples of “C₁₋₄alkylsulphonylaminocarbonyl” include methylsulphonylaminocarbonyl, isopropylsulphonylaminocarbonyl and t-butylsulphonylaminocarbonyl. Examples of “C₁₋₄alkylsulphonyl” include methylsulphonyl, isopropylsulphonyl and t-butylsulphonyl.

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate. fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

Within the present invention it is to be understood that a compound of the formula (I) or a salt thereof may exhibit the phenomenon of tautomerism and that the formulae drawings within this specification can represent only one of the possible tautomeric forms. It is to be understood that the invention encompasses any tautomeric form which inhibits DNA gyrase and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names.

It will be appreciated by those skilled in the art that certain compounds of formula (I) contain an asymmetrically substituted carbon and/or sulphur atom, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which form possesses properties useful in the inhibition of DNA gyrase, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DNA gyrase by the standard tests described hereinafter.

It is also to be understood that certain compounds of the formula (I) and salts thereof can exist in solvated as well as unsolvated forms such as, for example, hydrated forms. It is to be understood that the invention encompasses all such solvated forms which inhibit DNA gyrase.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

R¹ is C₁₋₄alkyl.

R¹ is methyl.

R² is halo.

R² is chloro.

R³ is halo.

R³ is chloro.

W is —O—.

W is —N(R⁶)—.

W is —NH—.

W is —C(R⁷)(R⁸)—.

Ring A is cyclohexyl wherein the dashed bond is a single bond.

Ring A is cyclohexenyl wherein the dashed bond is a double bond.

X is a direct bond.

X is —CH₂—.

X is —C(O)—.

X is S(O)_{q}— (wherein q is 1 or 2).

Ring D is heterocyclyl; wherein if said heterocyclyl contains an —NH— moiety that nitrogen may be optionally substituted by a group selected from R⁹.

Ring D is pyridinyl, benzothiazolyl, quinolinyl, pyrimidinyl or thiazolyl.

Ring D is carbocyclyl.

Ring D is phenyl.

R⁴ is a substituent on carbon and is selected from hydroxy or C₁₋₄alkoxy.

R⁴ is a substituent on carbon and is selected from hydroxy or methoxy.

R⁵ is a substituent on carbon and is selected from carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or heterocyclyl-R¹¹—; wherein R¹¹ is —C(O)—.

R⁵ is a substituent on carbon and is selected from carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or morpholino-R¹¹—; wherein R¹¹ is —C(O)—.

R⁵ is a substituent on carbon and is selected from carboxy, N-(methoxy)carbamoyl, mesyl, methoxycarbonyl, ethoxycarbonyl, mesylamino or morpholinocarbonyl.

R⁶ is hydrogen

R⁶ is C₁₋₄alkyl.

n is 0 or 1.

m is 1 or 2; wherein the values of R⁵ may be the same or different.

Therefore in a further aspect of the invention there is provided a compound of formula (I) (as depicted above) wherein:

R¹ is C₁₋₄alkyl;

R² is halo;

R³ is halo;

W is —N(R⁶)—;

Ring A is cyclohexyl or cyclohexenyl wherein the dashed bond is a single or a double bond;

X is a direct bond;

Ring D is carbocyclyl;

R⁴ is a substituent on carbon and is selected from hydroxy or C₁₋₄alkoxy;

R⁵ is a substituent on carbon and is selected from carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or heterocyclyl-R¹¹—; wherein R¹¹ is —C(O)—;

R⁶ is hydrogen;

n is 0 or 1;

m is 1 or 2; wherein the values of R⁵ may be the same or different; or a pharmaceutically acceptable salt thereof.

Therefore in a further aspect of the invention there is provided a compound of formula (I) (as depicted above) wherein:

R¹ is methyl;

R² is chloro;

R³ is chloro;

W is —NH—;

Ring A is cyclohexyl or cyclohexenyl wherein the dashed bond is a single or a double bond;

X is a direct bond;

Ring D is phenyl;

R⁴ is a substituent on carbon and is selected from hydroxy or methoxy;

R⁵ is a substituent on carbon and is selected from carboxy, N-(methoxy)carbamoyl, mesyl, methoxycarbonyl, ethoxycarbonyl, mesylamino or morpholinocarbonyl;

n is 0 or 1;

m is 1 or 2; wherein the values of R⁵ may be the same or different;

or a pharmaceutically acceptable salt thereof.

In a further aspect of the invention is provided a compound of formula I which is a compound of formula IA:

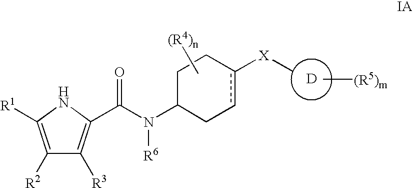

wherein:

R¹ is selected from hydrogen, halo, or C₁₋₄alkyl;

R² is selected from hydrogen, halo, or C₁₋₄alkyl;

R³ is selected from hydrogen, halo, or C₁₋₄alkyl;

“—” is a bond or is absent;

X is a direct bond, —CH₂—, —C(O)— or S(O)_{q}— (wherein q is 1 or 2);

Ring D is carbocyclyl or heterocyclyl; wherein if said heterocyclyl contains an —NH— moiety that nitrogen may be optionally substituted by a group selected from R⁹;

R⁴ and R⁵ are substituents on carbon and are independently selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N′,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N—(C₁₋₄alkyl)sulpharnoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁰— or heterocyclyl-R¹¹—; wherein R⁴ and R⁵ independently of each other may be optionally substituted on carbon by one or more R¹²; and wherein if said heterocyclyl contains an —NH— moiety that nitrogen may be optionally substituted by a group selected from R¹³;

R⁶, R⁷ and R⁸ are independently selected from hydrogen or C₁₋₄alkyl;

n is 0-4; wherein the values of R⁴ may be the same or different;

m is 0-4; wherein the values of R⁵ may be the same or different;

R¹² is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹⁴— or heterocyclyl-R¹⁵—; wherein R¹² independently of each other may be optionally substituted on carbon by one or more R¹⁶; and wherein if said heterocyclyl contains an —NH— moiety that nitrogen may be optionally substituted by a group selected from R¹⁷;

R⁹, R¹³ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;

R¹⁰, R¹¹, R¹⁴ and R¹⁵ are independently selected from a direct bond, —O—, —N(R¹⁸)—, —C(O)—, —N(R¹⁹)C(O)—, —C(O)N(R²⁰)—, —S(O)ₚ—, —SO₂N(R²¹)— or —N(R²²)SO₂—; wherein R¹⁸, R¹⁹, R²⁰, R²¹ and R²² are independently selected from hydrogen or C₁₋₄alkyl and p is 0-2;

R¹⁶ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

In a further aspect of the invention is provided a compound of formula IA which is a compound of formula IB:

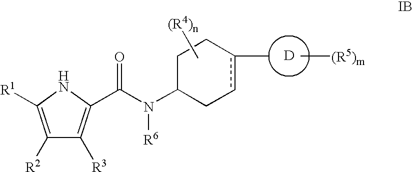

wherein:

R⁴ is a substituent on carbon and is selected from hydroxy, C₁₋₄alkoxy, carboxy, N—(C₁₋₄alkoxy)carbamoyl, C₁₋₄alkylS(O)ₐ wherein a is 2, C₁₋₄alkoxycarbonyl, or C₁₋₄alkylsulphonylamino; and

R⁵ is a substituent on carbon and is selected from carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or heterocyclyl-C(O)—.

In a further aspect of the invention is provided a compound of formula IB which is a compound of formula IC:

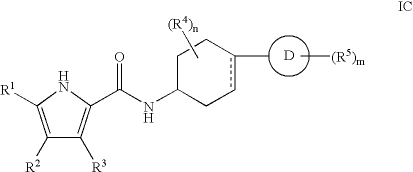

wherein:

Ring D is heterocyclyl; wherein if said heterocyclyl contains an —NH— moiety that nitrogen may be optionally substituted by a group selected from R⁹;

In a further aspect of the invention is provided a compound of formula IC which is a compound of formula ID:

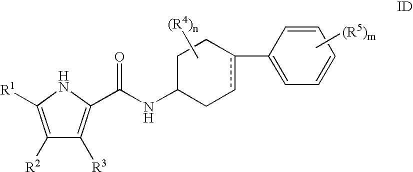

wherein

R⁴ is a substituent on carbon and is selected from hydroxy, C₁₋₄alkoxy, carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or heterocyclyl-R¹¹—; wherein R¹¹ is —C(O)—;

R⁵ is a substituent on carbon and is selected from carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or morpholino-C(O)—.

What is also provided is a compound of formula IE:

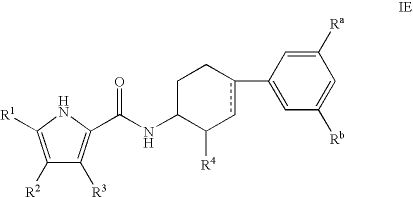

wherein:

R^{5s} is selected from carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or morpholino-C(O)—; and

R^{5b} is H or as defined for R^{5a}.

Particular compounds of the invention are the compounds of the Examples, each of which provides a further independent aspect of the invention. In further aspects, the present invention also comprises any two or more compounds of the Examples.

In one embodiment of the invention are provided compounds of formula (I), in an alternative embodiment are provided pharmaceutically-acceptable salts of compounds of formula (I).

In a further aspect the present invention provides a process for preparing a compound of formula (I) or a pharmaceutically-acceptable salt thereof.

Thus, the present invention also provides that the compounds of the formula (I) and pharmaceutically-acceptable salts thereof, can be prepared by a process as follows (wherein the variables are as defined above unless otherwise stated):
Process a) for compounds of formula (I) wherein W is —C(R⁷)(R⁸)—; converting a compound of formula (II):

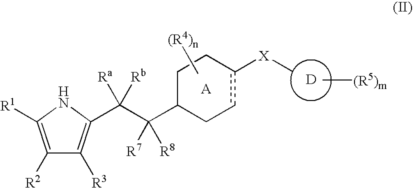

wherein R^{a} is cyano and R^{b} is dimethyamino or diethylamino; or R^{a} and R^{b} are independently selected from C₁₋₄alkylthio; or R^{a} and R^{b} together form 1,3-dithianyl or 1,3-dithiolanyl; into a compound of formula (I);
Process b) for compounds of formula (I) wherein W is —O—; reacting a compound of formula (III):

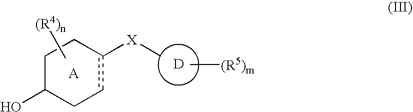

with a compound of formula (IV):

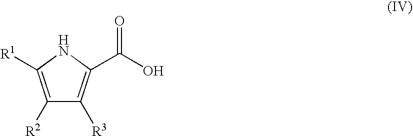

Process c) for compounds of formula (I) wherein W is —N(R⁶)—; reacting a compound of formula (V):

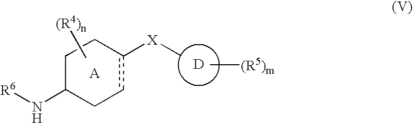

with a compound of formula (IV) or an activated acid derivative thereof;
Process d) for compounds of formula (I) wherein W is —C(R⁷)(R⁸)—; reacting a compound of formula (VI):

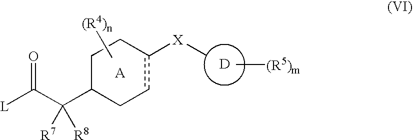

herein L is a displaceable group; with a compound of formula (VII):

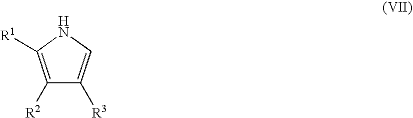

Process e) for compounds of formula (I) wherein W is —C(R⁷)(R⁸)—; reacting a compound of formula (VIII):

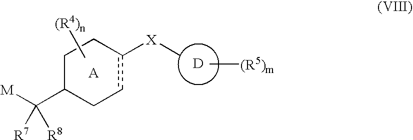

wherein M is an organometallic group; with a compound of formula (IX):

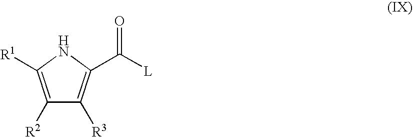

wherein L is a displaceable group;
Process f) for compounds of formula (I) wherein Ring A is cyclohexenyl; reacting a compound of formula (X):

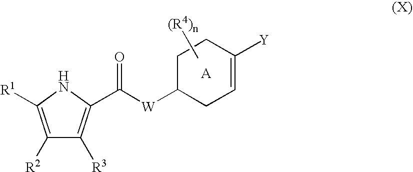

with a compound of formula (XI):

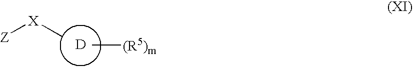

wherein one of Y and Z is an organometallic group and the other is a displaceable group;
Process g) for compounds of formula (I) wherein Ring A is cyclohexyl, by reducing a compound of formula (I) wherein Ring A is cyclohexenyl;
Process h) for compounds of formula (I) wherein Ring A is cyclohexenyl, by dehydrating a compound of formula (XII);

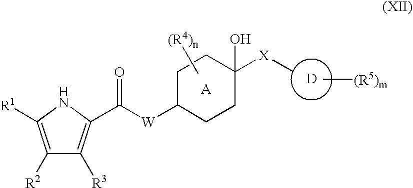

and thereafter if necessary:
i) converting a compound of the formula (I) into another compound of the formula (I);
ii) removing any protecting groups;
iii) forming a pharmaceutically acceptable salt.

L is a displaceable group. Suitable values for L include halo, for example chloro and bromo, pentafluorophenoxy and 2,5-oxopyrrolidin-1-yloxy.

M is an organometallic group, suitable values for M include organocuprates, for example CuLi, organozincs, Zn, or a Grignard reagent for example MgG where G is halo for example chloro.

One of X and Y is an organometallic group suitable values for this organometallic group include an organoborate, for example B(OR^{a}) wherein R^{a} is hydrogen or C₁₋₄alkyl, organocuprates, for example CuLi, organozincs, Zn, or a Grignard reagent for example MgG where G is halo for example chloro.

One of X and Y is a displaceable group suitable values for this displaceable group include halo, for example chloro and bromo, tosyl or mesylate.

Specific reaction conditions for the above reaction are as follows.
Process a) Compounds of formula (II) may be converted into compounds of formula (I):
(i) where R^{a} is cyano and R^{b} is dimethyamino or diethylamino; in the presence of a base for example sodium hydroxide, in a suitable solvent for example aqueous methanol at room temperature.
(ii) wherein or R^{a} and R^{b} are independently selected from C₁₋₄alkylthio; or R^{a} and R^{b} together form 1,3-dithianyl or 1,3-dithiolanyl; in the presence of a reagent such as a mercury, copper or silver salt for example Hg(ClO₄)₂, CuCl₂ or AgNO₃/Ag₂O in the presence of a suitable solvent for example methanol, acetone or ethanol from a temperature ranging from room temperature to reflux.

Compounds of formula (II) may be prepared according to Scheme 1:

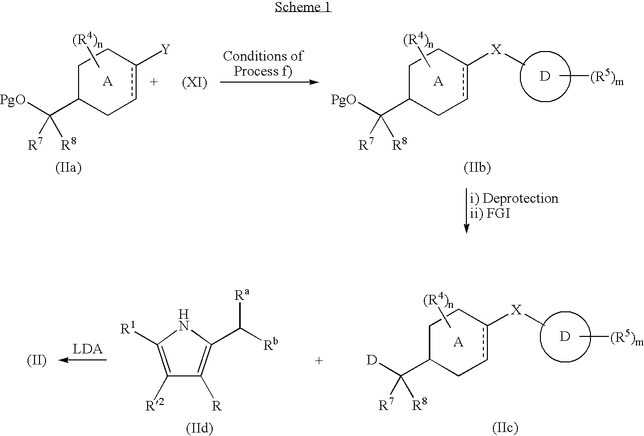

wherein Pg is a hydroxy protecting group as defined hereinbelow; one of Y and Z is an organometallic group as defined hereinabove and the other is a displaceable group as defined hereinabove; and D is a displaceable group. Suitable values for D include halo, for example chloro, bromo and iodo, tosylate and mesylate.

Deprotection of hydroxy protecting groups are well known in the art. Examples of such deprotections are given hereinbelow.

FGI stands for Functional Group Interconversion. In the above scheme such conversions between a hydroxy group and a D group are well known in the art and are well within the capabilities of a person skilled in the art.

Compounds of formula (IIa) and (IId) are known in the literature, or they are prepared by standard processes known in the art.
Process b) Compounds of formula (Ill) and (IV) may be reacted together in the presence of a coupling reagent, for example dicyclohexylcarbodiimide or EDC, in a suitable solvent, for example dichloromethane, THF or diethylether.

Compounds of formula (III) may be prepared according to Scheme 2:

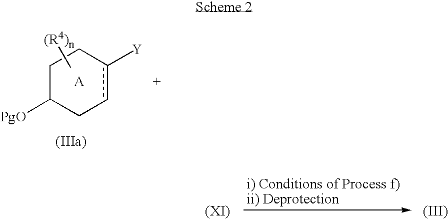

wherein Pg is a hydroxy protecting group as defined hereinbelow; and one of Y and Z is an organometallic group and the other is a displaceable group as defined hereinabove.

Deprotection of hydroxy protecting groups are well known in the art. Examples of such deprotections are given hereinbelow.

Compounds of formula (IIIa) and (IV) are commercially available compounds, or they are known in the literature, or they are prepared by standard processes known in the art.
Process c) Compounds of formula (V) and (IV) may be coupled together in the presence of a suitable coupling reagent. Standard peptide coupling reagents known in the art can be employed as suitable coupling reagents, or for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Compounds of formula (V) may be prepared according to Scheme 3:

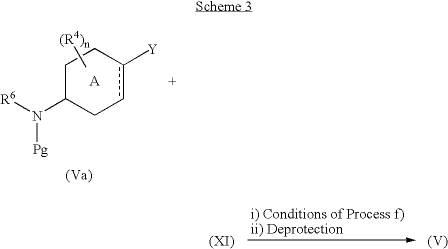

wherein Pg is a amino protecting group as defined hereinbelow; and one of Y and Z is an organometallic group and the other is a displaceable group as defined hereinabove. The skilled reader will appreciate that where R⁶ is hydrogen, this hydrogen also needs protecting by way of a suitable protecting group.

Deprotection of amino protecting groups are well known in the art. Examples of such deprotections are given hereinbelow.

Compounds of formula (Va) are commercially available compounds, or they are known in the literature, or they are prepared by standard processes known in the art.
Process d) Compounds of formula (VI) and (VII) may be reacted in a suitable solvent such a DCM or 1,2-dichloroethane, optionally in the presence of a Lewis acid, for example AlCl₃, from 0° C. to room temperature.

Compounds of formula (VI) may be prepared according to Scheme 4:

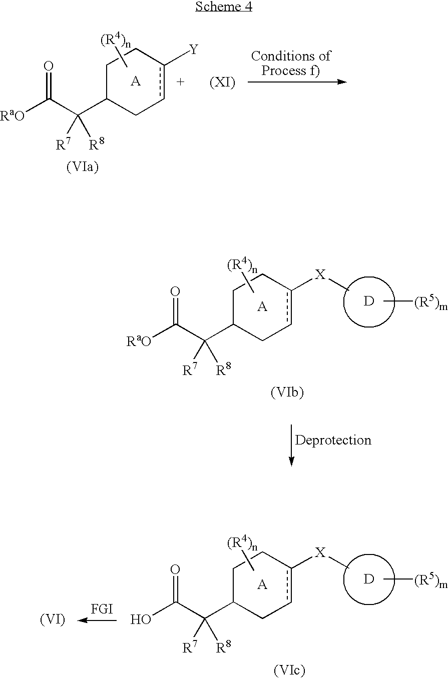

wherein R^{a}OC(O) is an ester group; and one of Y and Z is an organometallic group and the other is a displaceable group as defined hereinabove.

Suitable values for R^{a} include C₁₋₆alkyl. Deprotection of the R^{a} carboxy protecting group may be achieved under standard conditions, for example acid or base hydrolysis, such as those conditions give hereinbelow.

FGI stands for Functional Group Interconversion. In the above scheme such conversions between an acid group and a —C(O)L group are well known in the art and are well within the capabilities of a person skilled in the art.

Compounds of formula (VIa) and (VII) are commercially available compounds, or they are known in the literature, or they are prepared by standard processes known in the art.
Process e) Compounds of formula (VIII) and (IX) may be reacted in a suitable aprotic solvent such as THF or ether, at temperatures in the range of −78° C. to 0° C.

Compounds of formula (VIII) may be prepared from compounds of formula (IIc) under standard conditions known in the art. For example where M is an organocuprous reagent such compounds could be prepared according to Scheme 5:

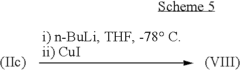

Compounds of formula (IX) are commercially available compounds, or they are known in the literature, or they are prepared by standard processes known in the art.
Process f) Compounds of formula (X) and (XI) may be reacted in a suitable aprotic solvent such as THF or ether, at temperatures in the range of −78° C. to 0° C.

Compounds of formula (X) may be prepared according to Scheme 6:

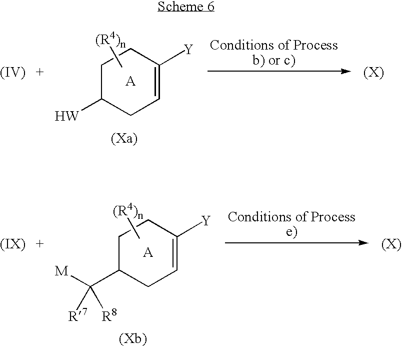

wherein M is an organometallic group as defined hereinabove.

Compounds of formula (Xa), (Xb) and (XI) are commercially available compounds, or they are known in the literature, or they are prepared by standard processes known in the art.
Process g) Compounds of formula (I) wherein Ring A is cyclohexenyl may be reduced under standard reduction conditions for example palladium catalyzed hydrogenation.
Process h) Compounds of formula (XII) may be dehydrated under acidic conditions, for example trifluoroacetic acid or hydrochloric acid or under basic conditions, for example using a base such as sodium hydroxide at temperatures in the range of 25° C.-50 ° C.

Compounds of formula (XII) may be prepared by one of Process a)-e) hereinabove.

The formation of a pharmaceutically-acceptable salt is within the skill of an ordinary organic chemist using standard techniques.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. The reagents used to introduce such ring substituents are either commercially available or are made by processes known in the art.

Introduction of substituents into a ring may convert one compound of the formula (I) into another compound of the formula (I). Such reactions and modifications include, for example, introduction of a substituent by means of an aromatic substitution reaction, reduction of substituents, alkylation of substituents, oxidation of substituents, esterification of substituents, amidation of substituents, formation of heteroaryl rings. The reagents and reaction conditions for such procedures are well known in the chemical art. Particular examples of aromatic substitution reactions include the introduction of alkoxides, diazotization reactions followed by introduction of thiol group, alcohol group, halogen group. Examples of modifications include; oxidation of alkylthio to alkylsulphinyl or alkylsulphonyl.

The skilled organic chemist will be able to use and adapt the information contained and referenced within the above references, and accompanying Examples therein and also the Examples herein, to obtain necessary starting materials, and products. If not commercially available, the necessary starting materials for the procedures such as those described above may be made by procedures which are selected from standard organic chemical techniques, techniques which are analogous to the synthesis of known, structurally similar compounds, or techniques which are analogous to the above described procedure or the procedures described in the examples. It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art, or they may be removed during a later reaction step or work-up.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

### Enzyme Potency Testing Methods

Compounds were tested for inhibition of GyrB ATPase activity using an ammonium molybdate/malachite green-based phosphate detection assay (Lanzetta, P. A., L. J. Alvarez, P. S. Reinach, and O. A. Candia, 1979, 100: 95-97). Assays were performed in multiwell plates in 100 μl reactions containing: 50 mM TRIS buffer pH 7.5, 75 mM ammonium acetate, 5.5 mM magnesium chloride, 0.5 mM ethylenediaminetetraacetic acid, 5% glycerol, 1 mM 1,4-Dithio-DL-threitol, 200 nM bovine serum albumin, 16 μg/ml sheared salmon sperm DNA, 4 nM E. coli GyrA, 4 nM E. coli GyrB, 250 μM ATP, and compound in dimethylsulfoxide. Reactions were quenched with 150 μl of ammonium molybdate/malachite green detection reagent containing 1.2 mM malachite green hydrochloride, 8.5 mM ammonium molybdate tetrahydrate, and 1 M hydrochloric acid. Plates were read in an absorbance plate reader at 625 nm and percent inhibition values were calculated using dimethylsulfoxide (2%)-containing reactions as 0% inhibition and novobiocin-containing (2 μM) reactions as 100% inhibition controls. Compounds were tested for inhibition of topoisomeraseIV ATPase activity as described above for GyrB except the 100 μl reactions contained the following: 20 mM TRIS buffer pH 8, 50 mM ammonium acetate, 8 mM magnesium chloride, 5% glycerol, 5 mM 1,4-Dithio-DL-threitol, 0.005% Brij-35, 5 μg/ml sheared salmon sperm DNA, 10 nM E. coli GyrA, 10 nM E. coli GyrB, 160 μM ATP, and compound in dimethylsulfoxide. Compound potency was based on IC₅₀ measurements determined from reactions performed in the presence of 10 different compound concentrations.

Compounds of the Examples generally have an IC₅₀ of <20 μg/ml.

### Bacterial Susceptibility Testing Methods

Compounds were tested for antimicrobial activity by susceptibility testing in liquid media. Compounds were dissolved in dimethylsulfoxide and tested in 10 doubling dilutions in the susceptibility assays. The organisms used in the assay were grown overnight on suitable agar media and then suspended in a liquid medium appropriate for the growth of the organism. The suspension was a 0.5 McFarland and a further 1 in 10 dilution was made into the same liquid medium to prepare the final organism suspension in 100 μL. Plates were incubated under appropriate conditions at 37 degrees C. for 24 hrs prior to reading. The Minimum Inhibitory Concentration was determined as the lowest drug concentration able to reduce growth by 80% or more.

Example 16 had an MIC of 1 μg/ml against Streptococcus pneumoniae. Other examples are provided in the following table.

| Example No. | MIC SPN548 | MIC SAU516 | MIC HIN446 |
|---|---|---|---|
| 5 | 4 | 4 | 16 |
| 11 | 0.13 | 0.5 | 2 |
| 16 | 1 | 2 | 4 |

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt thereof.

According to a further feature of the invention there is provided a method for inhibition of bacterial DNA gyrase and/or topoisomeraseIV in a warm-blooded animal, such as a human being, in need of such treatment which comprises administering to said animal an effective amount of a compound of formula (I) or a pharmaceutically acceptable salt thereof as defined hereinbefore.

According to a further feature of the invention there is provided a method of treating a bacterial infection in a warm-blooded animal, such as a human being, in need of such treatment which comprises administering to said animal an effective amount of a compound of formula (I) or a pharmaceutically acceptable salt thereof as defined hereinbefore.

A further feature of the present invention is a compound of formula (I) and pharmaceutically acceptable salts thereof for use as a medicament. Suitably the medicament is an antibacterial agent.

According to a further aspect of the invention there is provided the use of a compound of formula (I), or a pharmaceutically acceptable salt thereof in the manufacture of a medicament for use in the production of an anti-bacterial effect in a warm-blooded animal such as a human being.

According to a further aspect of the invention there is provided the use of a compound of formula (I), or a pharmaceutically acceptable salt thereof in the manufacture of a medicament for use in inhibition of bacterial DNA gyrase and/or topoisomeraseIV in a warm-blooded animal such as a human being.

Thus according to a further aspect of the invention there is provided the use of a compound of formula (I), or a pharmaceutically acceptable salt thereof in the manufacture of a medicament for use in the treatment of a bacterial infection in a warm-blooded animal such as a human being.

According to a further aspect of the invention there is provided a compound of formula (I), or a pharmaceutically acceptable salt thereof for use in the production of an anti-bacterial effect in a warm-blooded animal such as a human being.

According to a further aspect of the invention there is provided a compound of formula (I), or a pharmaceutically acceptable salt thereof for use in inhibition of bacterial DNA gyrase and/or topoisomeraseIV in a warm-blooded animal such as a human being.

Thus according to a further aspect of the invention there is provided a compound of formula (I), or a pharmaceutically acceptable salt thereof for use in the treatment of a bacterial infection in a warm-blooded animal such as a human being.

In order to use a compound of the formula (I) or a pharmaceutically-acceptable salt thereof, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition that comprises a compound of the formula (I) or a pharmaceutically-acceptable salt thereof, and a pharmaceutically-acceptable diluent or carrier.

According to a further aspect of the invention there is provided a pharmaceutical composition that comprises a compound of formula (I) as defined hereinbefore or a pharmaceutically acceptable salt thereof, in association with a pharmaceutically acceptable excipient or carrier for use in producing an anti-bacterial effect in an warm-blooded animal, such as a human being.

According to a further aspect of the invention there is provided a pharmaceutical composition that comprises a compound of formula (I) as defined hereinbefore or a pharmaceutically acceptable salt thereof, in association with a pharmaceutically acceptable excipient or carrier for use in inhibition of bacterial DNA gyrase and/or topoisomeraseIV in an warm-blooded animal, such as a human being.

According to a further aspect of the invention there is provided a pharmaceutical composition that comprises a compound of formula (I) as defined hereinbefore or a pharmaceutically acceptable salt thereof, in association with a pharmaceutically acceptable excipient or carrier for use in the treatment of a bacterial infection in an warm-blooded animal, such as a human being.

In order to use a compound of the formula (I) or a pharmaceutically-acceptable salt thereof, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the formula (I) or a pharmaceutically-acceptable salt thereof, and a pharmaceutically-acceptable diluent or carrier.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in producing an anti-bacterial effect in an warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in inhibition of bacterial DNA gyrase in an warm-blooded animal, such as a human being.

According to a further aspect of the invention there is provided a pharmaceutical composition which comprises a compound of formula (I) as defined hereinbefore or a pharmaceutically acceptable salt thereof, in association with a pharmaceutically acceptable excipient or carrier for use in the treatment of a bacterial infection in an warm-blooded animal, such as a human being.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

In addition to the compounds of the present invention the pharmaceutical composition of this invention may also contain or be co-administered (simultaneously, sequentially or separately) with one or more known drugs selected from other clinically useful antibacterial agents (for example, macrolides, quinolones, β-lactams or aminoglycosides) and/or other anti-infective agents (for example, an antifungal triazole or amphotericin). These may include carbapenems, for example meropenem or imipenem, to broaden the therapeutic effectiveness. Compounds of this invention may also contain or be co-administered with bactericidal/permeability-increasing protein (BPI) products or efflux pump inhibitors to improve activity against gram negative bacteria and bacteria resistant to antimicrobial agents.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

In addition to its use in therapeutic medicine, compounds of formula (I) and their pharmaceutically acceptable salts are also useful as pharmacological tools in the development and standardization of in-vitro and in-vivo test systems for the evaluation of the effects of inhibitors of DNA gyrase in laboratory animals such as cats, dogs, rabbits, monkeys, rats and mice, as part of the search for new therapeutic agents.

In the above other, pharmaceutical composition, process, method, use and medicament manufacture features, the alternative and particular embodiments of the compounds of the invention described herein also apply.

### EXAMPLES

The invention will now be illustrated by the following non limiting examples in which, unless stated otherwise:
(i) temperatures are given in degrees Celsius (° C.); operations were carried out at room or ambient temperature, that is, at a temperature in the range of 18-25° C.;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii) in general, the course of reactions was followed by TLC and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 300 MHz using perdeuterio dimethyl sulphoxide (DMSO-d₆) as solvent unless otherwise indicated;
(vii) chemical symbols have their usual meanings; SI units and symbols are used;
(viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (M+H)⁺;
(x) where a synthesis is described as being analogous to that described in a previous example the amounts used are the millimolar ratio equivalents to those used in the previous example;
(xi) the following abbreviations have been used:

| | |
|---|---|
| THF | tetrahydrofuran; |
| HOBt | 1-hydroxybenzotriazole; |
| EDC | 1-(3-dimethylaminopropyl)-3-ethyl carbodiimide; |
| TFA | trifluoroacetic acid; |
| DCM | dichloromethane; |
| DMF | N,N-dimethylformamide; and |
| DMSO | dimethylsulphoxide. |

### Example 1

### trans-Ethyl 3-(4-{[(3,4-dichloro-5-methyl-1H-pyrrol-2-yl)carbonyl]amino}cyclohexyl)benzoate

To a stirred solution of trans-ethyl 3-(4-aminocyclohexyl)benzoate (Intermediate 12; 280 mg, 0.911 mmol), 3,4-dichloro-5-methyl-1H-pyrrole-2-carboxylic acid (Intermediate 28; 172 mg, 0.911), HOBt (123 mg, 0.911 mmol) and N-methylmorpholine (0.5 ml, 4.56 mmol) in methylene chloride (5 ml) was added EDC (314 mg, 1.64 mmol). The reaction mixture was stirred overnight and then partitioned between DCM and water. The organic layer was washed with 1 N HCl, saturated sodium bicarbonate, and saturated sodium chloride, dried over sodium sulfate, filtered, and concentrated under reduced pressure. The crude residue was purified by flash column chromatography (1.5:1 hexanes/ethyl acetate) to provide the title compound (117 mg, 30%). NMR: 1.32 (t, 3H), 1.51 (m, 4H), 1.83 (br d, 2H), 1.99 (br d, 2H), 2.13-2.30 (m, 4H), 2.63 (m, 2H), 3.84 (m, 1H), 4.31 (q, 2H), 7.14 (d, 1H), 7.46 (dd, 1H), 7.58 (d, 1H), 7.70-7.94 (m, 2H), 11.99 (br s, 1H); MS m/z 423, 425.

### Examples 2-4

The following compounds were prepared by the procedure of Example 1 starting from 3,4-dichloro-5-methyl-1H-pyrrole-2-carboxylic acid (Intermediate 28) and the indicated starting material (SM).

| Ex | Compound | Data | SM |
|---|---|---|---|
| 2 | cis-Ethyl 3-(4-{[(3,4-dichloro- | MS m/z 424, 426 | Intermediate |
| | 5-methyl-1H-pyrrol-2-yl) | | 13 |
| | carbonyl]amino}cyclohexyl) | | |
| | benzoate | | |
| 3 | Ethyl 3-(4-{[(3,4-dichloro-5- | NMR (CD₃OD): 1.43 (t, 3H), 1.77 (m, | Intermediate |
| | methyl-1H-pyrrol-2- | 4H), 2.18 (m, 4H), 2.26 (s, 3H), 4.27 | 15 |
| | yl)carbonyl]amino}-1- | (m, 1H), 4.41 (q, 2H), 7.50 (t, 1H), 7.76- | |
| | hydroxycyclohexyl)benzoate | 7.86 (m, 1H), 7.93, dd, 1H), 8.22 (s, | |
| | | 1H); MS m/z 440, 442 | |
| 4 | Dimethyl 5-(4-{[(3,4- | NMR: 1.87-2.00 (m, 1H), 2.08-2.22 (m, | Intermediate |
| | dichloro-5-methyl-1H-pyrrol- | 1H), 2.23-2.48 (m, 1H), 2.52-2.72 (m, | 16 |
| | 2-yl)carbonyl] amino} | 3H), 3.70-3.87 (m, 1H), 3.95 (s, 6H), | |
| | cyclohex-1-en-1- | 6.17 (m, 1H), 8.23 (s, 1H), 8.36 (s, 1H), | |
| | yl)isophthalate | 8.55 (s, 1H); MS m/z 465, 467 | |

### Example 5

### trans-3-(4-{[(3,4-Dichloro-5-methyl-1H-pyrrol-2-yl)carbonyl]amino}cyclohexyl)benzoic acid

To a stirred solution of trans-ethyl 3-(4-{[(3,4-dichloro-5-methyl-1H-pyrrol-2-yl)carbonyl]amino}cyclohexyl)benzoate (Example 1; 90 mg, 0.213 mmol) in 2:1:1 THF/ethanol/water (6 ml) was added lithium hydroxide (11 mg, 0.255 mmol). The reaction mixture was stirred overnight and then partitioned between ethyl acetate and water. The aqueous layer was acidified to pH 3 by addition of 10% HCl. The solid that formed was collected by filtration to give the title compound (36 mg, 43%). NMR: 1.31-1.72 (m, 4H), 1.88 (br d, 2H), 2.01 (br d, 2H), 2.18 (s, 3H), 2.61 (t, 1H), 3.85 (dd, 1H), 7.15 (d, 1H), 7.42 (t, 1H), 7.46-7.63 (m, 1H), 7.64-7.93 (m, 2H), 12.00 (s, 1H), 12.92 (br s, 1H); MS m/z 396, 398.

### Examples 6-8

The following compounds were prepared by the procedure of Example 5 starting from the indicated starting material (SM).

| Ex | Compound | Data | SM |
|---|---|---|---|
| 6 | cis-3-(4-{[(3,4-Dichloro- | NMR: 1.69-1.81 (m, 6H), 1.90 (m, 2H), 2.15 (s, | Example |
| | 5-methyl-1H-pyrrol-2- | 3H), 2.65 (m, 1H), 4.20-4.25 (m, 1H), 7.15 (m, | 2 |
| | yl)carbonyl]amino} | 1H), 7.29-7.34 (m, 1H), 7.39-7.42 (m, 1H), 7.75- | |
| | cyclohexyl)benzoic acid | 7.77 (m, 1H), 7.86 (s, 1H); MS m/z 396, 398 | |
| 7 | 3-(4-{[(3,4-Dichloro-5- | NMR: 1.43-1.71 (m, 4H), 2.03 (m, 4H), 2.20 (s, | Example |
| | methyl-1H-pyrrol-2- | 3H), 4.12 (m, 1H), 7.35 (d, 1H), 7.49 (t, 1H), 7.80 | 3 |
| | yl)carbonyl]amino}-1- | (dd, 2H), 12.06 (s, 1H), 12.94 (s, 1H); MS m/z | |
| | hydroxycyclohexyl) | 412, 414 | |
| | benzoic acid | | |
| 8 | 5-(4-{[(3,4-Dichloro-5- | NMR: 1.87-2.00 (m, 1H), 2.08-2.22 (m, 1H), | Example |
| | methyl-1H-pyrrol-2- | 2.23-2.48 (m, 1H), 2.52-2.72 (m, 3H), 3.70-3.87 | 4 |
| | yl)carbonyl]amino}cyclohex- | (m, 1H), 6.17 (m, 1H), 8.23 (s, 1H), 8.36 (s, 1H), | |
| | 1-en-1-yl)isophthalic | 8.55 (s, 1H), 12.01 (s, 1H); MS m/z 437, 439 | |
| | acid | | |
| 9 | 3-(trans-4-{[(3,4- | M.p. 254° C. NMR: 1.87 (m, 1H), 2.00 (m, 1H), | Example |
| | Dichloro-5-methyl-1H- | 2.19 (s, 3H), 2.51 (m, 2H), 3.38 (s, 3H), 4.08 (m, | 12 |
| | pyrrol-2-yl)carbonyl] | 2H), 6.21 (m, 1H), 7.33 (d, 1H), 7.47 (t, 1H), 7.72 | |
| | amino}-3-methoxy | (d, 1H), 7.86 (d, 1H), 8.01 (s, 1H), 12.14 (s, 1H). | |
| | cyclohex-1-en-1- | MS (ESN): 423.24 (M⁻) for C₂₀H₂₀Cl₂N₂O₄ | |
| | yl)benzoic acid | | |

### Example 10

### 3,4-Dichloro-N-(4-{3-[(methoxyamino)carbonyl]phenyl}cyclohex-3-en-1-yl)-5-methyl-1H-pyrrole-2-carboxamide

To a solution of 5-(4-{[(3,4-dichloro-5-methyl-1H-pyrrol-2-yl)carbonyl]amino}cyclohex-1-en-1-yl)benzoic acid (Example 16; 37 mg, 0.09 mmol), methoxyamine hydrochloride (6 mg, 0.09 mmol), HOBt (12 mg, 0.09 mmol), and N-methylmorpholine (0.3 ml, 0.27 mmol) in DCM (3 ml) was added EDC (31 mg, 0.16 mmol). The reaction mixture was stirred overnight and then partitioned between ethyl acetate and water. The organic layer was washed with 1 N HCl, saturated sodium bicarbonate, and saturated sodium chloride, dried over sodium sulfate, filtered, and concentrated under reduced pressure to give the title compound (19 mg, 48%). NMR: 1.61-1.82 (m, 4H), 2.07-2.10 (m, 4H), 2.24 (s, 3H), 3.77 (s, 3H), 4.16 (m, 1H), 7.35 (d, 1H), 7.47-7.55 (m, 1H), 7.63 (d, 1H), 7.74 (d, 1H), 7.97 (s, 1H), 11.80 (s, 1H), 12.11 (s, 1H); MS m/z 441, 443.

### Example 11

### 3-((3,4-Cis)-4-{[(3,4-Dichloro-5-methyl-1H-pyrrol-2-yl)carbonyl]amino}-3-methoxycyclohex-1-en-1-yl)benzoic acid

N-[(1,2)-cis-4-Bromo-2-methoxycyclohex-3-en-1-yl]-3,4-dichloro-5-methyl-1H-pyrrole-2-carboxamide (Intermediate 26; 100 mg, 0.262 mmol), (3-ethoxycarbonylphenyl)boronic acid (61 mg, 0.31 mmol), PS-PPh₃-Pd (resin, 0.11 mmol/g, 240 mg, 0.026 mmol) and potassium carbonate (127 mg, 0.92 mmol) were suspended in a mixture of DME/EtOH/H₂O (2:2:1, 4 ml) in a sealed tube. The reaction mixture was heated to 150° C. under microwave conditions, cooled down to room temperature and filtered. The filter cake was washed with ethyl acetate (10 ml) and water (5 ml), the combined aqueous layer was acidified to pH 2 with 2M HCl, and extracted with ethyl acetate (2×10 ml), the combined organic layer was dried, concentrated under vacuum and purified by column chromatography (5% to 15% gradient MeOH in ethyl acetate) giving the title compound as an off white solid (6 mg). M.p. 255° C. NMR: 1.87 (m, 2H), 2.19 (s, 3H), 2.51 (m, 2H), 3.42 (s, 3H), 4.05 (m, 1H), 4.27 (m, 1H), 6.31 (m, 1H), 7.12 (d, 1H), 7.50 (t, 1H), 7.78 (d, 1H), 7.85 (d, 1H), 7.98 (s, 1H), 12.17 (br s, 1H), 13.05 (br s, 1H). MS (ESN): 423.24 (M⁻) for C₂₀H₂₀Cl₂N₂O₄.

### Examples 12-15

The following compounds were prepared by the procedure of Example 11 starting from the indicated starting material (SM).

| Ex | Compound | Data | SM |
|---|---|---|---|
| 12 | Ethyl 3-(trans-4-{[(3,4- | M.p. 135° C. NMR: 1.33 (t, 3H), 1.88 (m, | Intermediate |
| | dichloro-5-methyl-1H- | 1H), 2.00 (m, 1H), 2.19 (s, 3H), 2.51 (m, | 27 and [3- |
| | pyrrol-2-yl)carbonyl] | 2H), 3.39 (s, 3H), 4.09 (m, 2H), 4.35 (q, | (ethoxy |
| | amino}-3-methoxy | 2H), 6.23 (m, 1H), 7.25 (d, 1H), 7.52 (t, 1H), | carbonyl) |
| | cyclohex-1-en-1- | 7.78 (d, 1H), 7.88 (d, 1H), 8.00 (s, 1H), | phenyl] |
| | yl)benzoate | 12.04 (s, 1H); MS (ESN): 451.31 (M⁻) | boronic acid |
| | | for C₂₂H₂₄Cl₂N₂O₄ | |
| 13 | 3,4-Dichloro-N-(trans- | M.p. 205° C. NMR: 1.88 (m, 1H), 2.00 (m, | Intermediate |
| | 2-methoxy-4-{3- | 1H), 2.18 (s, 3H), 2.51 (m, 2H), 2.99 (s, 3H), | 27 and 3- |
| | [(methylsulfonyl) | 3.37 (s, 3H), 4.06 (m, 1H), 4.08 (m, 1H), | [(methylsul- |
| | amino]phenyl} | 6.11 (m, 1H), 7.15 (d, 1H), 7.21~7.35 (m, | fonyl)amino] |
| | cyclohex-3-en-1-yl)-5- | 4H), 9.74 (s, 1H), 12.04 (s, 1H); MS (ESN): | phenylboronic |
| | methyl-1H-pyrrole-2- | 472.26 (M⁻) for C₂₀H₂₃Cl₂N₃O₄S | acid |
| | carboxamide | | |
| 14 | 3,4-Dichloro-N-{trans- | M.p. 120° C. NMR: 1.86 (m, 1H), 1.99 (m, | Intermediate |
| | 2-methoxy-4-[3- | 1H), 2.19 (s, 3H), 2.51 (m, 2H), 3.38 (s, 3H), | 27 and 3- |
| | (morpholin-4- | 3.59 (m, 8H), 4.09 (m, 2H), 6.23 (m, 1H), | (morpholin-4- |
| | ylcarbonyl)phenyl] | 7.23 (d, 1H), 7.33 (d, 1H), 7.44 (t, 1H), 7.49 | ylcarbonyl) |
| | cyclohex-3-en-1-yl}-5- | (s, 1H), 7.58 (d, 1H), 12.04 (s, 1H); MS | phenylboronic |
| | methyl-1H-pyrrole-2- | (ESN): 492.31 (M⁻) for C₂₄H₂₇Cl₂N₃O₄. | acid |
| | carboxamide | | |
| 15 | 3,4-Dichloro-N-{trans- | M.p. 185° C. NMR: 1.88 (m, 1H), 2.00 (m, | Intermediate |
| | 2-methoxy-4-[3- | 1H), 2.19 (s, 3H), 2.51 (m, 2H), 3.26 (s, 3H), | 27 and 3- |
| | (methylsulfonyl) | 3.39 (s, 3H), 4.10 (m, 2H), 6.33 (m, 1H), | (methylsul- |
| | phenyl]cyclohex-3-en- | 7.26 (d, 1H), 7.67 (t, 1H), 7.86 (m, 2H), 7.98 | fonyl)phenyl- |
| | 1-yl}-5-methyl-1H- | (s, 1H), 12.04 (s, 1H); MS (ESN): 457.23 | boronic acid |
| | pyrrole-2-carboxamide | (M⁻) for C₂₀H₂₂Cl₂N₂O₄S | |

### Example 16

### 5-(4-{[(3,4-Dichloro-5-methyl-1H-pyrrol-2-yl)carbonyl]amino}cyclohex-1-en-1-yl)benzoic acid

To a solution of 3-(4-{[(3,4-dichloro-5-methyl-1H-pyrrol-2-yl)carbonyl]amino}-1-hydroxycyclohexyl)benzoic acid (Example 7; 40 mg, 0.972 mmol) was added trifuoroacetic acid (1 ml). The reaction mixture was stirred for 45 min, diluted with water, and the solid that formed was collected by filtration to give the title compound (25 mg, 60%). NMR: 1.83 (m, 2H), 1.98 (m, 2H), 2.09-2.36 (m, 5H), 4.10 (m, 1H), 6.19 (s, 1H), 7.24 (d, 1H), 7.47 (t, 1H), 7.70 (d, 1H), 7.82 (d, 1H), 7.97 (s, 1H), 12.03 (s, 1H); MS m/z 394, 396.

### Preparation of Starting Materials

### Intermediate 1

### Ethyl 3-(8-hydroxy-1,4-dioxaspiro[4.5]dec-8-yl)benzoate

To a −20° C. (dry ice, 2-propanol) solution of ethyl 3-iodobenzoate (3.31 g, 12.00 mmol) in THF (17 ml) was slowly added 2-propyl magnesium chloride (6.18 ml, 2.0 M in THF, 12.36 mmol). The reaction mixture was stirred for 30 min then 1,4-dioxaspiro[4.5]decan-8-one (1.50 g, 9.60 mmol) was added. The reaction mixture was warmed to 0° C. and stirred for 3 h. The reaction mixture was partitioned between ethyl acetate and saturated ammonium chloride. The organic layer was washed with saturated sodium chloride, dried over sodium sulfate, and concentrated under reduced pressure. The crude residue was purified by flash column chromatography (1:1 hexanes/ethyl acetate) to give the title compound (1.05 g, 36%). NMR (CDCl₃): 1.79 (m, 1H), 1.40 (t, 3H), 1.79 (m, 1H), 2.01 (m, 2H), 2.18 (m, 1H), 2.52 (m, 2H), 4.00 (m, 2H), 4.04 (s, 4H), 4.38 (q, 2H), 7.41 (t, 1H), 7.93 (m, 1H), 7.96 (dt, 1H), 8.21 (t, 1H).

### Intermediate 2

The following compound was prepared by the procedure of Intermediate 1 using the starting material indicated.

| Int | Compound | NMR (CDCl₃) | SM |
|---|---|---|---|
| 2 | Dimethyl 5-(8- | 1.77 (dd, 4H), 2.00-2.36 | Dimethyl 4- |
| | hydroxy-1,4- | (m, 4H), 3.95 (s, 6H), | iodoisophthalate |
| | dioxaspiro[4.5]dec- | 3.95 (s, 4H), 8.41 (s, 2H), | |
| | 8-yl)isophthalate | 8.58 (s, 1H) | |

### Intermediate 3

### Ethyl 3-(4-oxocyclohex-1-en-1-yl)benzoate

To a 0° C. solution of ethyl 3-(8-hydroxy-1,4-dioxaspiro[4.5]dec-8-yl)benzoate (Intermediate 1; 1.05 g, 3.43 mmol) was slowly added TFA (10 ml). The ice bath was removed and the reaction mixture was heated to 40° C. for 20 min. The reaction mixture was cooled to room temperature and then partitioned between chloroform and water. The organic layer was washed with saturated sodium bicarbonate and saturated sodium chloride, dried over sodium sulfate, filtered, and concentrated under reduced pressure. The crude residue was purified by flash column chromatography (1:1 hexanes/ethyl acetate) to give the title compound (395 mg, 50%). NMR (CDCl₃): 1.37 (t, 3H), 2.77 (m, 2H), 2.98 (m, 2H), 3.33 (m, 2H), 4.42 (q, 2H), 6.34 (s, 1H), 7.43 (t, 1H), 7.72 (d, 1H), 7.98 (d, 1H), 8.23 (s, 1H).

### Intermediate 4

The following compound was prepared by the procedure of Intermediate 3 using the starting material indicated.

| Int | Compound | NMR (CDCl₃) | SM |
|---|---|---|---|
| 4 | Dimethyl 5-(4- | 2.69 (t, 2H), 2.91-3.02 | Intermediate |
| | oxocyclohex-1-en- | (m, 2H), 3.04-3.20 (m, 2H), | 2 |
| | 1-yl)isophthalate | 3.97 (s, 6H), 6.27 (s, 1H), | |
| | | 8.26 (s, 2H), 8.59 (s, 1H) | |

### Intermediate 5

### Ethyl 3-(4-hydroxycyclohex-1-en-1-yl)benzoate

To a solution of ethyl 3-(4-oxocyclohex-1-en-1-yl)benzoate (Intermediate 3; 487 mg, 2.10 mmol) in 2-propanol (5 ml) was added sodium borohydride (87 mg, 2.31 mmol). The reaction mixture was stirred at room temperature for 1 h, methanol was added, and concentrated under reduced pressure. The crude residue was purified by flash column chromatography (2:1 hexanes/ethyl acetate) to give the title compound (236 mg, 48%). NMR (CDCl₃): 1.40 (t, 3H), 1.88 (m, 1H), 2.04 (m, 1H), 2.26 (m, 1H), 2.58 (m, 3H), 4.09 (m, 1H), 4.39 (q, 2H), 6.07 (s, 1H), 7.38 (t, 1H), 7.55 (d, 1H), 7.91 (d, 1H), 8.06 (s, 1H).

### Intermediate 6

The following compound was prepared by the procedure of Intermediate 5 using the starting material indicated.

| Int | Compound | NMR (CDCl₃) | SM |
|---|---|---|---|
| 6 | Dimethyl 5-(4- | 1.74-1.92 (m, 1H), 1.96-2.11 | Intermediate |
| | hydroxycyclohex-1- | (m, 1H), 2.17-2.35 (m, 1H), | 4 |
| | en-1-yl)isophthalate | 2.46-2.72 (m, 3H), 3.95 (s, 6H), | |
| | | 4.09 (m, 1H), 6.16 (m, 1H), | |
| | | 8.24 (s, 2H), 8.54 (m, 1H) | |

### Intermediate 7 and Intermediate 8

### cis-Ethyl 3-(4-hydroxycyclohexyl)benzoate and trans-ethyl-3-(4-hydroxycyclohexyl)benzoate

A mixture of ethyl 3-(4-hydroxycyclohex-1-en-1-yl)benzoate (Intermediate 5; 236 mg, 1.00 mmol), ammonium formate (635 mg, 10.00 mmol) and 10% Pd/C (10 mg) in ethanol (10 ml) was heated to 50° C. for 1 h. The reaction mixture was cooled to room temperature, filtered through diatomaceous earth, and concentrated under reduced pressure. The crude residue was purified by flash column chromatography (1.5:1 hexanes/ethyl acetate) to give cis-ethyl 3-(4-hydroxycyclohexyl)benzoate (100 mg, 42%) and trans-ethyl-3-(4-hydroxycyclohexyl)benzoate (57 mg, 25%).

cis-ethyl 3-(4-hydroxycyclohexyl)benzoate: NMR (CDCl₃): 1.40 (t, 3H), 1.70 (m, 4H), 1.91 (m, 4H), 2.61 (dt, 1H), 4.15 (m, 1H), 4.37 (q, 2H), 7.45-7.36 (m, 3H), 7.88 (d, 1H), 7.93 (s, 1H).

trans-ethyl-3-(4-hydroxycyclohexyl)benzoate: NMR (CDCl₃): 2.14 (br d, 2H), 2.57 (dt, 1H), 3.73 (dt, 1H), 4.37 (q, 2H), 7.41-7.38 (m, 2H), 7.89-7.86 (m, 2H).

### Intermediate 9

### trans-Ethyl 3-(4-azidocyclohexyl)benzoate

To a 0° C. solution of cis-ethyl 3-(4-hydroxycyclohexyl)benzoate (Intermediate 7; 318 mg, 1.29 mmol) in DCM (5 ml) and pyridine (0.15 ml) was added mesyl chloride (0.14 ml, 1.81 mmol). The reaction mixture was stirred for 20 min and then partitioned between DCM and 1 N HCl. The organic layer was washed with water and saturated sodium chloride, dried over sodium sulfate, filtered, and concentrated under reduced pressure. The crude residue was dissolved in DMSO (3 ml) and treated with tetrabutyl ammonium hydrogen sulfate (219 mg, 0.65 mmol) and sodium azide (252 mg, 3.87 mmol). The reaction mixture was heated to 50° C. for 72 h. The reaction mixture was partitioned between ethyl acetate and water. The organic layer was dried over sodium sulfate, filtered, and concentrated under reduced pressure. The crude residue was purified by flash column chromatography (1.5:1 hexanes/ethyl acetate) to give the title compound (251 mg, 71%). NMR (CDCl₃): 1.29-1.46 (m, 3H), 1.46-1.72 (m, 4H), 1.91-2.09 (m, 2H), 2.08-2.25 (m, 2H), 2.46-2.69 (m, 1H), 3.26-3.50 (m, 1H), 4.38 (q, 2H), 7.32-7.50 (m, 2H), 7.80-7.97 (m, 2H).

### Intermediates 10-11

The following compounds were prepared by the procedure of Intermediate 9 using the starting material indicated.

| Int | Compound | NMR (CDCl₃) | SM |
|---|---|---|---|
| 10 | Dimethyl 5-(4- | 1.85-2.00 (m, 1H), 2.08-2.22 (m, 1H), 2.24-2.45 (m, | Intermediate |
| | azidocyclohexyl) | 1H), 2.49-2.72 (m, 3H), 3.70-3.87 (m, 1H), 3.95 (s, | 6 |
| | isophthalate | 6H), 6.17 (m, 1H), 8.23 (m, 2H), 8.55 (s, 1H) | |
| 11 | cis-Ethyl 3-(4- | 1.40 (t, 3H), 1.62-1.89 (m, 6H), 1.89-2.12 (m, 2h), | Intermediate |
| | azidocyclohexyl) | 2.50-2.73 (m, 1H), 3.82-4.03 (m, 1H), 4.38 (q, 2H), | 8 |
| | benzoate | 7.30-7.51 (m, 2H), 7.80-7.98 (m, 2H) | |

### Intermediate 12

### trans-Ethyl 3-(4-aminocyclohexyl)benzoate

A mixture of trans-ethyl 3-(4-azidocyclohexyl)benzoate (Intermediate 9; 251 mg, 0.912 mmol), 10% Pd/C (25 mg) and acetic acid (0.5 ml) in ethanol (5 ml) was shaken under an atmosphere of hydrogen at 40 psi for 30 h. The reaction mixture was filtered through diatomaceous earth and concentrated under reduced pressure to provide the title compound (274 mg, 98%). NMR (CDCl₃): 1.40 (m, 5H), 1.60 (m, 2H), 1.98 (m, 2H), 2.02 (m, 2H), 2.60 (m, 1H), 3.09 (m, 1H), 4.37 (q, 2H), 6.41 (br s, 2H), 7.37 (m, 2H), 7.87 (m, 2H).

### Intermediate 13

The following compound was prepared by the procedure of Intermediate 12 using the starting material indicated.

| Int | Compound | NMR (CDCl₃) | SM |
|---|---|---|---|
| 13 | cis-Ethyl 3-(4- | 1.39 (t, 3H), 1.80-2.18 | Intermediate |
| | aminocyclohexyl) | (m, 8H), 2.65 (m, 1H), | 11 |
| | benzoate | 3.54 (m, 1H), 4.36 (q, 2H), | |
| | | 6.88 (br s, 2H), 7.36 | |
| | | (m, 2H), 7.52 (m, 1H), | |
| | | 7.87-7.93 (m, 2H) | |

### Intermediate 14

### Ethyl 3-(1-hydroxy-4-oxocyclohexyl)benzoate

A solution of ethyl 3-(8-hydroxy-1,4-dioxaspiro[4.5]dec-8-yl)benzoate (Intermediate 1; 1.51 g, 4.93 mmol) in ethanol (10 ml) was treated with 10% HCl (5 ml). The reaction mixture was stirred for 3 days and then concentrated under reduced pressure to give the title compound (1.0 g, 77%). NMR (CDCl₃): 1.38 (t, 3H), 1.56-1.88 (m, 2H), 2.34-2.46 (m, 4H), 2.88-3.02 (m, 2H), 4.43 (q, 2H), 7.43 (m, 1H), 7.78 (m, 1H), 7.99 (m, 1H), 8.24 (m, 1H).

### Intermediate 15

### Ethyl 3-(4-amino-1-hydroxycyclohexyl)benzoate

To a solution of ethyl 3-(1-hydroxy-4-oxocyclohexyl)benzoate (Intermediate 14; 1.0 g, 3.81 mmol) in methanol (9 ml) was added ammonium formate (2.4 g, 38.12 mmol) and water (1 ml). The mixture was stirred for 10 min followed by the addition of 10% Pd/C (400 mg). The reaction mixture was stirred overnight, filtered through diatomaceous earth, and concentrated under reduced pressure. The crude residue was purified by flash column chromatography (9:1:0.1 chloroform/methanol/concentrated ammonium hydroxide gave the title compound (312 mg, 31%). NMR (CDCl₃): 1.40 (t, 3H), 1.65-1.72 (m, 4H), 2.02-2.07 (m, 2H), 2.32-2.43 (m, 2H), 3.16-3.20 (m, 1H), 4.40 (q, 2H), 7.41-7.46 (m, 1H), 7.75 (d, 1H), 7.95 (d, 1H), 8.22 (s, 1H); MS m/z 264.

### Intermediate 16

### Ethyl 3-(4-aminocyclohexyl)benzoate

To a 0° C. solution of dimethyl 5-(4-azidocyclohexyl)isophthalate (Intermediate 10; 170 mg, 0.536 mmol) in THF (15 ml), water (1.5 ml), and 2 N sodium hydroxide (0.1 ml) was added trimethylphosphine (0.75 ml of a 1 M solution in THF, 0.749 mmol). The reaction mixture was stirred at room temperature overnight and then concentrated under reduced pressure. The crude residue was partitioned between ethyl acetate and water. The organic layer was dried over sodium sulfate, filtered, and concentrated under reduced pressure. The crude residue was dissolved in methanol and HCl in diethyl ether was added. The solid that formed was collected by filtration to give the hydrochloride salt of the title compound (56 mg, 31%). MS m/z 340.

### Intermediate 17

### 4-Bromo-2-methoxycyclohex-3-en-1-yl acetate

To a suspension of sodium hydride (484 mg, 20.16 mmol) in dry DMF (20 ml) at 0° C. was added tetra-n-butylamonium bromide (542 mg, 1.68m mol) and iodomethane (4.78 g, 33.6 mmol). The resulting mixture was treated dropwise with solution of (1,2-trans-)4-bromo-2-hydroxycyclohex-3-en-1-yl acetate (3.95 g, 16.8 mmol) (Aust. J. Chem., 1991, 44, 939-950) in DMF (10 ml), the mixture was then stirred at 0° C. for 3 h then was kept in a 4° C. refrigerator overnight. It was quenched with saturated aqueous NH₄Cl (10 ml) and the resulting mixture was poured into 5% HCl solution (100 ml) at 0° C., and extracted with ethyl acetate (100 ml×2), dried, concentrated and purified by flash column chromatography (50% DCM in hexanes) to give the title compound as a yellowish oil (diastereomeric mixture, 2.17 g). NMR (CDCl₃) (diastereomers): 1.82 (m, 2H), 2.08 (m, 2H), 2.08 (s, 3H), 2.12 (s, 3H), 2.54 (m, 2×2H), 3.43 (s, 2×3H), 3.72 (m, 1H), 3.86 (m, 1H), 5.12 (m, 1H), 5.15 (m, 1H), 6.11 (m, 1H), 6.13 (m, 1H).

### Intermediate 18 and Intermediate 19

### (1,2-trans)-4-Bromo-2-methoxycyclohex-3-en-1-ol and (1,2-cis)-4-Bromo-2-methoxycyclohex-3 -en-1-ol

4-Bromo-2-methoxycyclohex-3-en-1-yl acetate (Intermediate 17; 2.15 g, 8.64 mmol) was dissolved in THF/MeOH (1:1; 20 ml), it was then cooled down to 0° C. 1 N Sodium hydroxide (17.2 ml) was added and the resulting mixture was stirred at 0° C. for 20 min, followed by addition of 5% HCl (10 ml), the mixture was extracted with ethyl acetate (30 ml×2). The organic phase was washed with brine and dried, concentrated to provide an oil. This crude sample was further purified by flash column chromatography (ethyl acetate/hexanes/DCM, 1; 5:2) and afforded two diastereomers, the relative conformation was confirmed by NOE proton NMR experiment, the less polar one was cis-isomer (551 mg), while the polar one was the corresponding trans-isomer (460 mg). (1,2-trans)-4-Bromo-2-methoxycyclohex-3-en-1-ol: NMR (CDCl₃): 1.80 (m, 1H), 1.99 (m, 1H), 2.30 (m, 1H), 2.55 (m, 2H), 3.44 (s, 3H), 3.67 (m, 1H), 3.77 (m, 1H), 6.08 (m, 1H). (1,2-cis)-4-Bromo-2-methoxycyclohex-3-en-1-ol: NMR (CDCl₃): 1.78 (m, 1H), 2.00 (m, 1H), 2.42 (m, 2H), 2.63 (m, 1H), 3.46 (s, 3H), 3.72 (m, 1H), 3.97 (m, 1H), 6.10 (m, 1H).

### Intermediate 20

### (1,2-trans)-4-Bromo-2-methoxycyclohex-3-en-1-yl methanesulfonate

(1,2-trans)-4-Bromo-2-methoxycyclohex-3-en-1-ol (Intermediate 18; 460 mg, 2.22 mmol) was dissolved in dry DCM (10 ml), cooled down to 0° C., and treated with methanesulfonyl chloride (382 mg, 3.33 mmol) along with triethylamine (448 mg, 4.44 mmol). The mixture was stirred at 0° C. for 10 min, and then warmed up to room temperature, stirred for another 30 min. The reaction mixture was diluted with ethyl acetate (25 ml), washed with water and brine, dried and concentrated to give an oil as the desired product. The weight of the product was not measured and it was used in the next step without further purification. NMR (CDCl₃): 1.80 (m, 1H), 1.99 (m, 1H), 2.30 (m, 1H), 2.55 (m, 2H), 3.44 (s, 3H), 3.67 (m, 1H), 3.77 (m, 1H), 6.08 (m, 1H).

### Intermediate 21

The following compound was prepared by the procedure of Intermediate 20 using the starting material indicated.

| Int | Compound | NMR (CDCl₃) | SM |
|---|---|---|---|
| 21 | (1,2-cis)-4-Bromo-2- | 1.99 (m, 1H), 2.33 | Intermediate |
| | methoxycyclohex-3-en- | (m, 1H), 2.54 (m, 2H), | 19 |
| | 1-yl methanesulfonate | 3.09 (s, 3H), 3.45 (s, 3H), | |
| | | 3.91 (m, 1H), 5.08 (m, 1H), | |
| | | 6.10 (m, 1H) | |

### Intermediate 22

### (1,6-cis)-6-Azido-3-bromocyclohex-2-en-1-yl methyl ether

(1,2-trans)-4-Bromo-2-methoxycyclohex-3-en-1-yl methanesulfonate (Intermediate 20; crude) was dissolved in dimethyl sulfoxide (5 ml), treated with sodium azide (519 mg, 7.98 mmol), and stirred at 65° C. for 10 days. Although the reaction was still not completed, it was cooled down to room temperature and diluted with DCM (30 ml), washed with water and brine, the organic layer was collected and dried. It was then purified by column chromatography (ethyl acetate/hexanes/DCM, 1:2:4) and gave the desired product as an oil (157 mg). NMR (CDCl₃): 1.85 (m, 1H), 2.13 (m, 1H), 2.50 (m, 1H), 2.62 (m, 1H), 3.49 (s, 3H), 3.67 (m, 1H), 3.85 (m, 1H), 6.16 (m, 1H).

### Intermediate 23

The following compound was prepared by the procedure of Intermediate 22 using the starting material indicated.

| Int | Compound | NMR (CDCl₃) | SM |
|---|---|---|---|
| 23 | (1,6-trans)-6-Azido- | 1.81 (m, 1H), 1.99 (m, 1H), | Intermediate |
| | 3-bromocyclohex-2-en- | 2.48 (m, 1H), 2.70 (m, 1H), | 21 |
| | 1-yl methyl ether | 3.48 (s, 3H), 3.61 (m, 1H), | |
| | | 3.91 (m, 1H), 6.10 (m, 1H) | |

### Intermediate 24

### (1,2-cis)-4-Bromo-2-methoxycyclohex-3-en-1-amine

To a solution of (1,6-cis)-6-azido-3-bromocyclohex-2-en-1-yl methyl ether (Intermediate 22; 157 mg, 0.68 mmol) in THF/H₂O (12:1, 13 ml), 2M sodium hydroxide (0.5 ml) was added, the mixture was cooled down to 0° C., followed by the addition of trimethyl phosphine (77 mg, 1.02 mmol). The resulting mixture was stirred at 0° C., warmed up to room temperature, and stirred overnight. DCM (50 ml) was added, the mixture was stirred for 5 min, the aqueous layer was extracted with DCM (2×20 ml), the combined organic phase was dried, concentrated under vacuum to give the title compound as an oil (127 mg). NMR (CDCl₃): 1.70 (m, 1H), 1.87 (m, 1H), 2.50 (m, 2H), 3.05 (m, 1H), 3.42 (s, 3H), 3.63 (m, 1H), 6.17 (m, 1H).

### Intermediate 25

The following compound was prepared by the procedure of Intermediate 24 using the starting material indicated.

| Int | Compound | NMR (CDCl₃) | SM |
|---|---|---|---|
| 25 | (1,2-trans)-4- | 1.80 (m, 1H), 1.87 | Intermediate |
| | Bromo-2- | (m, 1H), 2.51 (m, 2H), | 23 |
| | methoxycyclohex- | 2.92 (m, 1H), 3.41 | |
| | 3-en-1-amine | (s, 3H), 3.50 (m, 1H), | |
| | | 6.11 (m, 1H) | |

### Intermediate 26

### N-[(1,2)-cis-4-Bromo-2-methoxycyclohex-3-en-1-yl]-3,4-dichloro-5-methyl-1H-pyrrole-2-carboxamide

(1,2-cis)-4-Bromo-2-methoxycyclohex-3-en-1-amine (Intermediate 24; 125 mg, 0.61 mmol), 5-methyl-3,4-dichloropyrolyl carboxylic acid (141 mg, 0.73 mmol), HOBt (262 mg, 1.94 mmol) and N-methyl-morpholine (216 mg, 2.14 mmol) were mixed in dry DCM (10 ml) and cooled down to 0° C. 1-(3-Dimethylaminopropyl)-3-ethylcarbodiimide hydrochloride (175 mg, 0.92 mmol) was added, the mixture was stirred for 10 minutes. 3,4-dichloro-5-methyl-1H-pyrrole-2-carboxylic acid (Intermediate 28; 125 mg, 0.61 mmol) was added, the mixture was stirred for over night, diluted with 50 ml of DCM and washed with 5% HCl and brine. The organic phase was dried and concentrated under vacuum. The residue was dissolved in DCM (6 ml), heated, and hexane was added and the resulting precipitate was filtered and collected as the desired product (220 mg). NMR (CDCl₃): 1.87 (m, 1H), 2.04 (m, 1H), 2.26 (s, 3H), 2.59 (m, 2H), 3.45 (s, 3H), 3.73 (m, 1H), 4.28 (m, 1H), 6.31 (m, 1H), 7.27 (m1H), 9.83 (br, 1H). MS (ESN): 381.16 (M−1) for C₁₄H₁₅Cl₂N₂O₂Br.

### Intermediate 27

The following compound was prepared by the procedure of Intermediate 26 using the starting material indicated.

| Int | Compound | NMR | SM |
|---|---|---|---|
| 27 | N-[(1,2)-trans-4-Bromo-2- | 1.87 (m, 2H), 2.18 (s, 3H), 2.51 (m, | Intermediate |
| | methoxycyclohex-3-en-1- | 2H), 3.31 (s, 3H), 3.91 (m, 1H), 4.03 | 25 and |
| | yl]-3,4-dichloro-5-methyl- | (m, 1H), 6.18 (m, 1H), 7.23 (d, 1H), | Intermediate |
| | 1H-pyrrole-2-carboxamide | 12.02 (br, 1H); MS (ESN): 381.16 | 28 |
| | | (M − 1) for C₁₄H₁₅Cl₂N₂O₂Br | |

### Intermediate 28

### 3,4-Dichloro-5-methyl-1H-pyrrole-2-carboxylic acid

Ethyl 3,4-dichloro-5-methyl-1H-pyrrole-2-carboxylate (Intermediate 29, 7.765 g, 0.03496 mol) was dissolved in MeOH (80 ml) and DCM (10 ml) and slowly added to a 70° C. solution of 2 N LiOH (105 ml, 0.21 mol). After 2 h, the reaction mixture was cooled to room temperature and then in an ice bath, followed by acidification with 2 N HCl. The mixture was stirred at 0° C. for 1 h, and a purple solid was filtered, washed with water and lyophilized overnight to give 4.314 g (0.0222 mol, 64% yield) of the desired product. NMR: 2.17 (s, 3H); MS (ES⁻): 192.13, 194.13 (M−H)⁻ for C₆H₅Cl₂NO₂.

### Intermediate 29

### Ethyl 3,4-dichloro-5-methyl-1H-pyrrole-2-carboxylate

A solution of ethyl 5-methyl-1H-pyrrole-2-carboxylate (Intermediate 30; 7.00 g, 0.0457 mol) in tetrachloromethane (30 ml) was cooled to 0° C. under nitrogen. The rubber septum used in the apparatus was pierced with a needle, and SO₂Cl₂ (7.8 ml, 0.096 mol) was then added dropwise over 25 minutes. Within 1 hr, the reaction mixture had formed a slurry. The solid was collected by suction filtration, washed with cold tetrachloromethane, and dried under vacuum overnight to give the title product as a peach coloured solid (7.84 g, 0.0353 mol, 77% yield). NMR: 1.34-1.40 (t, 3H), 2.28 (s, 3H), 4.32-4.38 (m, 2H); MS (ES⁻): 222.00, 224.00 (M−H)⁻ for C₈H₉Cl₂NO₂.

### Intermediate 30

### Ethyl 5-methyl-1H-pyrrole-2-carboxylate

Sodium (2.79 g, 0.121 mmol) was dissolved in anhydrous EtOH (100 ml), then 2,2,2-trichloro-1-(5-methyl-1H-pyrrol-2-yl)ethanone (Intermediate 31; 22.5 g, 0.099 mmol) was added in small portions. The dark brown solution was stirred at room temperature for 30 minutes then concentrated under vacuum to a small volume. The mixture was cooled in an ice bath and 3 N HCl was added slowly then extracted with diethyl ether (3×100 ml). The ether extracts were washed with 10% NaHCO₃, water and brine, dried over Na₂SO₄ and concentrated in vacuo to give the title compound as a brown solid. (15.04 g). NMR: 1.32 (t, 3H), 2.10 (s, 3H), 4.37 (q, 2H), 5.96 (dd, 1H), 6.78 (dd, 1H), 11.67 (s, 1H).

### Intermediate 31

### 2,2,2-Trichloro-1-(5-methyl-1H-pyrrol-2-yl)ethanone

2-Methyl-1H-pyrrole (Intermediate 32, 10 g, 0.123 mmol) in anhydrous diethyl ether (30 ml) was added dropwise over 1 h to a stirred solution of triacetyl chloride (29 g, 0.16 mmol) in anhydrous Et₂O (100 ml). The mixture was stirred for a further 1 h then K₂CO₃ (10 g/30 ml) was added slowly through a dropping funnel. The organic phase was dried over Na₂SO₄ and treated with decolourizing charcoal (3 g) for 30 minutes at room temperature. The resulting purple solution was concentrated and triturated with n-hexanes to give the title compound as a purple solid. (16.72 g). NMR (CDCl₃): 2.36 (s, 3H), 6.04 (dd, 1H), 7.45 (dd, 1H), 10.34 (s, 1H).

### Intermediate 32

### 2-Methyl-1H-pyrrole

Potassium hydroxide (50 g, 0.89 mmol) was added to a solution of ethylene glycol (750 ml) and 1H-pyrrole-2-carbaldehyde (50 g,0.53 mmol). Hydrazine hydrate (37 ml, 0.745 mmol) was added slowly over 15 minutes. The reaction mixture was refluxed at 90° C. for 90 minutes. The mixture was cooled to room temperature and cold water (250 ml) was added. The aqueous mixture was extracted with DCM (250 ml). The organic phase was washed with water (250 ml), dried over Na₂SO₄ and concentrated in vacuo. Kugelrohr distillation gave the title compound as a clear colourless liquid (29.75 g). NMR: 2.14 (s, 3H), 5.77 (s, 1H), 5.93 (dd, 1H), 6.25 (dd, 1H), 10.54 (s, 1H).

1. A compound of formula (I): 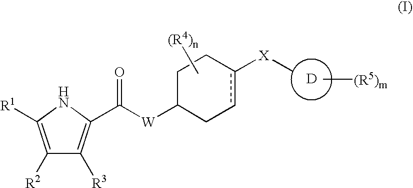
wherein:
R¹ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R¹ may be optionally substituted on carbon by one or more halo or cyclopropyl;
R² is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R² may be optionally substituted on carbon by one or more halo or C₃₋₆cycloalkyl;
R³ is selected from hydrogen, nitro, hydroxy, halo, cyano, C₁₋₄alkyl, C₁₋₄alkoxy, C₂₋₄alkenyl, C₂₋₄alkynyl, C₁₋₄alkanoyl, C₁₋₄alkylS(O)ₐ wherein a is 0 to 2 and C₃₋₆cycloalkyl; wherein R³ may be optionally substituted on carbon by one or more halo or C₃₋₆cycloalkyl;
W is —O—, —N(R⁶)— or —C(R⁷)(R⁸)—;
“—” is a bond or is absent;
X is a direct bond, —CH₂—, —C(O)— or S(O)_{q}— (wherein q is 1 or 2);
Ring D is carbocyclyl or heterocyclyl; wherein if said heterocyclyl contains an —NH— moiety that nitrogen may be optionally substituted by a group selected from R⁹;
R⁴ and R⁵ are substituents on carbon and are independently selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N—(C₁₋₄alkyl)sulphamoyl, N,N—(C₁₋₄alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁰— or heterocyclyl-R¹¹—; wherein R⁴ and R⁵ independently of each other may be optionally substituted on carbon by one or more R¹²; and wherein if said heterocyclyl contains an —NH— moiety that nitrogen may be optionally substituted by a group selected from R¹³;
R⁶, R⁷ and R⁸ are independently selected from hydrogen or C₁₋₄alkyl;
n is 0-4; wherein the values of R⁴ may be the same or different;
m is 0-4; wherein the values of R⁵ may be the same or different;
R¹² is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹⁴— or heterocyclyl-R¹⁵—; wherein R¹² independently of each other may be optionally substituted on carbon by one or more R¹⁶; and wherein if said heterocyclyl contains an —NH— moiety that nitrogen may be optionally substituted by a group selected from R¹⁷;
R⁹, R¹³ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;
R¹⁰, R¹¹, R¹⁴ and R¹⁵ are independently selected from a direct bond, —O—, —N(R¹⁸)—, —C(O)—, —N(R¹⁹)C(O)—, —C(O)N(R²⁰)—, —S(O)ₚ—, —SO₂N(R²¹)— or —N(R²²)SO₂—; wherein R¹⁸, R¹⁹, R²⁰, R²¹ and R²² are independently selected from hydrogen or C₁₋₄alkyl and p is 0-2;
R¹⁶ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof.

2. A compound of formula (I) as claimed in claim 1 which is a compound of formula IA: 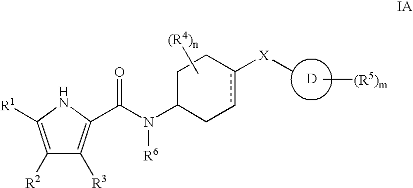
wherein:
R¹ is selected from hydrogen, halo, or C₁₋₄alkyl;
R² is selected from hydrogen, halo, or C₁₋₄alkyl;
R³ is selected from hydrogen, halo, or C₁₋₄alkyl;
“—” is a bond or is absent;
X is a direct bond, —CH₂—, —C(O)— or S(O)_{q}— (wherein q is 1 or 2);
Ring D is carbocyclyl or heterocyclyl; wherein if said heterocyclyl contains an —NH— moiety that nitrogen may be optionally substituted by a group selected from R⁹;
R⁴ and R⁵ are substituents on carbon and are independently selected from halo, nitro, cyano, hydroxy, trifluoromethoxy, amino, carboxy, carbamoyl, mercapto, sulphamoyl, sulfo, formyl, ureido, hydroxyiminomethyl, N-hydroxyformamido, hydrazinocarbonyl, N-hydroxyethanimidoyl, amino(hydroxyimino)methyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, N—(C₁₋₄alkoxy)carbamoyl, N′—(C₁₋₄alkyl)ureido, N,N′—(C₁₋₄alkyl)₂ureido, N—(C₁₋₄alkyl)-N—(C₁₋₄alkoxy)carbamoyl, C₁₋₄alkylS(O)ₐ wherein a is 0 to 2, C₁₋₄alkoxycarbonyl, C₁₋₄alkoxycarbonylamino, N—(C₁₋₄alkyl)sulphamoyl, N,N—(C_{1 4}alkyl)₂sulphamoyl, C₁₋₄alkylsulphonylamino, C₁₋₄alkylsulphonylaminocarbonyl, N′—(C₁₋₄alkyl)hydrazinocarbonyl, N′,N′—(C₁₋₄alkyl)₂hydrazinocarbonyl, carbocyclyl-R¹⁰— or heterocyclyl-R¹¹—; wherein R⁴ and R⁵ independently of each other may be optionally substituted on carbon by one or more R¹²; and wherein if said heterocyclyl contains an —NH— moiety that nitrogen may be optionally substituted by a group selected from R¹³;
R⁶, R⁷ and R⁸ are independently selected from hydrogen or C₁₋₄alkyl;
n is 0-4; wherein the values of R⁴ may be the same or different;
m is 0-4; wherein the values of R⁵ may be the same or different;
R¹² is selected from halo, nitro, cyano, hydroxy, trifluoromethoxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, C₁₋₄alkoxycarbonylamino, carbocyclyl-R¹⁴— or heterocyclyl-R¹⁵—; wherein R¹² independently of each other may be optionally substituted on carbon by one or more R¹⁶; and wherein if said heterocyclyl contains an —NH— moiety that nitrogen may be optionally substituted by a group selected from R¹⁷;
R⁹, R¹³ and R¹⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)carbamoyl, benzyl, benzyloxycarbonyl, benzoyl and phenylsulphonyl;
R¹⁰, R¹¹, R¹⁴ and R¹⁵ are independently selected from a direct bond, —O—, —N(R¹⁸)—, —C(O)—, —N(R¹⁹)C(O)—, —C(O)N(R²⁰)—, —S(O)ₚ—, —SO₂N(R²¹)— or —N(R²²)SO₂—; wherein R¹⁸, R¹⁹, R²⁰, R²¹ and R²² are independently selected from hydrogen or C₁₋₄alkyl and p is 0-2;
R¹⁶ is selected from halo, nitro, cyano, hydroxy, trifluoromethoxy, trifluoromethyl, amino, carboxy, carbamoyl, mercapto, sulphamoyl, methyl, ethyl, ethenyl, ethynyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof.

3. A compound of formula (I) as claimed in claim 1 which is a compound of formula IB: 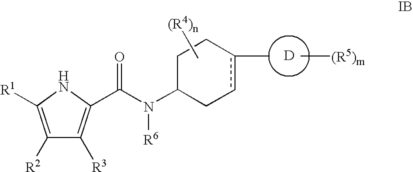
wherein:
R¹, R², R³, R⁶, n, “—”, Ring D and m are as defined in claim 1;
R⁴ is a substituent on carbon and is selected from hydroxy, C₁₋₄alkoxy, carboxy, N—(C₁₋₄alkoxy)carbamoyl, C₁₋₄alkylS(O)ₐ wherein a is 2, C₁₋₄alkoxycarbonyl, or C₁₋₄alkylsulphonylamino; and
R⁵ is a substituent on carbon and is selected from carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or heterocyclyl-C(O)—.

4. A compound of formula (I) as claimed in claim 1 which is a compound of formula IC: 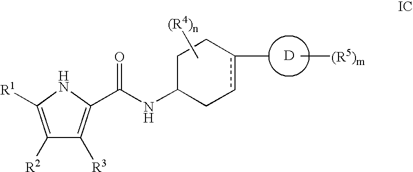
wherein:
R¹, R², R³, R⁴, n, “—”, R⁵ and m are as defined in claim 1; and
Ring D is heterocyclyl; wherein if said heterocyclyl contains an —NH— moiety that nitrogen may be optionally substituted by a group selected from R⁹;

5. A compound of formula (I) as claimed in claim 1 which is a compound of formula ID: 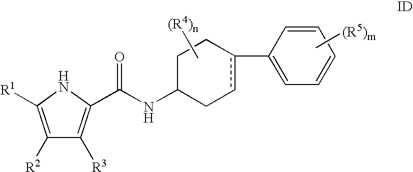
wherein
R¹, R², R³, n, “—” and m are as defined in claim 1:
R⁴ is a substituent on carbon and is selected from hydroxy, C₁₋₄alkoxy, carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or heterocyclyl-R¹¹—; wherein R¹¹ is —C(O)—; and
R⁵ is a substituent on carbon and is selected from carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or morpholino-C(O)—.

6. A compound of formula (I) as claimed in claim 1 which is a compound of formula IE: 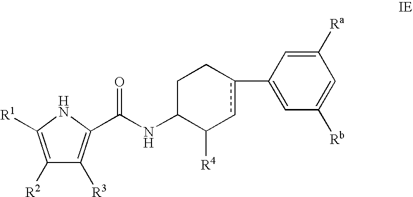
wherein:
R¹, R², R³, R⁴ and “—” as defined in claim 1;
R^{a} is selected from carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or morpholino-C(O)—; and
R^{b} is H or as defined for R^{b}.

7. A compound which is
trans-Ethyl 3-(4-{[(3,4-dichloro-5-methyl-1H-pyrrol-2-yl)carbonyl]amino}cyclohexyl)benzoate;
cis-Ethyl 3-(4-{[(3,4-dichloro-5-methyl-1H-pyrrol-2-yl)carbonyl]amino}cyclohexyl)benzoate;
Ethyl 3-(4-{[(3,4-dichloro-5-methyl-1H-pyrrol-2-yl)carbonyl]amino}-1-hydroxycyclohexyl)benzoate;
Dimethyl 5-(4-{[(3,4-dichloro-5-methyl-1H-pyrrol-2-yl)carbonyl]amino}cyclohex-1-en-1-yl)isophthalate;
trans-3-(4-{[(3,4-Dichloro-5-methyl-1H-pyrrol-2-yl)carbonyl]amino}cyclohexyl)benzoic acid;
cis-3-(4-{[(3,4-Dichloro-5-methyl-1H-pyrrol-2-yl)carbonyl]amino}cyclohexyl)benzoic acid;
3-(4-{[(3,4-Dichloro-5-methyl-1H-pyrrol-2-yl )carbonyl]amino}-1-hydroxycyclohexyl)benzoic acid;
5-(4-{[(3,4-Dichloro-5-methyl-1H-pyrrol-2-yl)carbonyl]amino}cyclohex-1-en-1-yl)isophthalic acid;
3-(trans-4-{[(3,4-Dichloro-5-methyl-1H-pyrrol-2-yl)carbonyl]amino}-3-methoxy cyclohex-1-en-1-yl)benzoic acid;
3,4-Dichloro-N-(4-{3-[(methoxyamino)carbonyl]phenyl}cyclohex-3-en-1-yl )-5-methyl-1H-pyrrole-2-carboxamide;
3-((3,4-Cis)-4-{[(3,4-Dichloro-5-methyl-1H-pyrrol-2-yl)carbonyl]amino}-3-methoxycyclohex-1-en-1-yl)benzoic acid;
Ethyl 3-(trans-4-{[(3,4-dichloro-5-methyl-1H-pyrrol-2-yl)carbonyl]amino}-3-methoxy cyclohex-1-en-1-yl)benzoate;
3,4-Dichloro-N-(trans-2-methoxy-4-{3-[(methylsulfonyl)amino]phenyl}cyclohex-3-en-1-yl)-5-methyl-1H-pyrrole-2-carboxamide;
3,4-Dichloro-N-{trans-2-methoxy-4-[3-(morpholin-4-ylcarbonyl)phenyl]cyclohex-3-en-1-yl}-5-methyl-1H-pyrrole-2-carboxamide;
3,4-Dichloro-N-{trans-2-methoxy-4-[3-(methylsulfonyl)phenyl]cyclohex-3-en-1-yl}-5-methyl-1H-pyrrole-2-carboxamide;
5-(4-{[(3,4-Dichloro-5-methyl-1H-pyrrol-2-yl)carbonyl]amino}cyclohex-1-en-1-yl)benzoic acid;
or a pharmaceutically acceptable salt thereof.

8. A pharmaceutical composition comprising a compound of claim 1 or a pharmaceutically-acceptable salt thereof, and a pharmaceutically-acceptable diluent or carrier.

9. (canceled)

10. A method for inhibiting bacterial DNA gyrase in a warm-blooded animal, such as a human being, in need of such treatment which comprises administering to said animal an effective amount of a compound of of claim 1 or a pharmaceutically acceptable salt.

11-13. (canceled)

14. A process for preparing a compound of claim 1, comprising:
Process a) for compounds of formula (I) wherein W is —C(R⁷)(R⁸)—; converting a compound of formula (II): 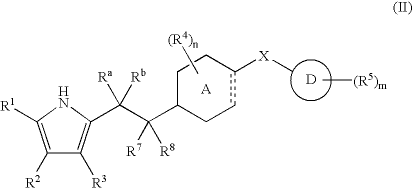
wherein R^{a} is cyano and R^{b} is dimethyamino or diethylamino; or R^{a} and R^{b} are independently selected from C₁₋₄alkylthio; or R^{a} and R^{b} together form 1,3-dithianyl or 1,3-dithiolanyl; into a compound of formula (I);
Process b) for compounds of formula (I) wherein W is —O—; reacting a compound of formula (III): 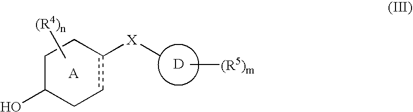
with a compound of formula (IV): 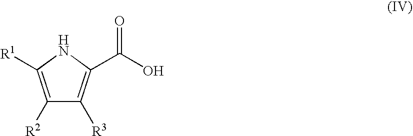
Process c) for compounds of formula (I) wherein W is -N(R⁶)-; reacting a compound of formula (V): 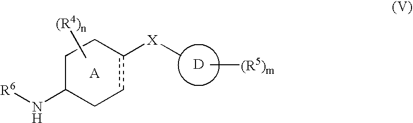
with a compound of formula (IV) or an activated acid derivative thereof;
Process d) for compounds of formula (I) wherein W is —C(R⁷)(R⁸)—; reacting a compound of formula (VI): 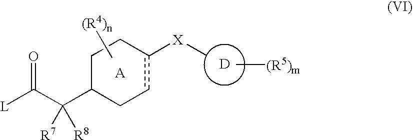
wherein L is a displaceable group; with a compound of formula (VII): 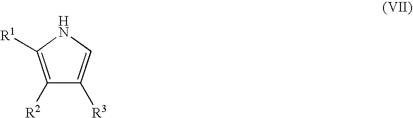
Process e) for compounds of formula (I) wherein W is —C(R⁷)(R⁸)—; reacting a compound of formula (VIII): 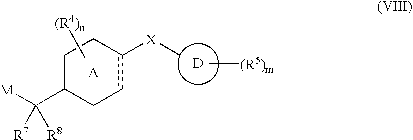
wherein M is an organometallic group; with a compound of formula (IX): 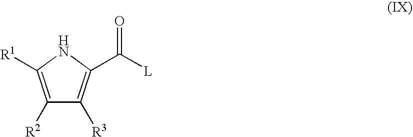
wherein L is a displaceable group;
Process f) for compounds of formula (I) wherein Ring A is cyclohexenyl; reacting a compound of formula (X): 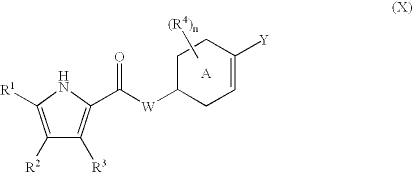
with a compounds of formula (XI): 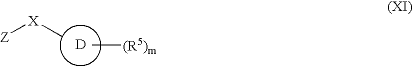
wherein one of Y and Z is an organometallic group and the other is a displaceable group;
Process g) for compounds of formula (I) wherein Ring A is cyclohexyl, by reducing a compound of formula (I) wherein Ring A is cyclohexenyl;
Process h) for compounds of formula (I) wherein Ring A is cyclohexenyl, by dehydrating a compound of formula (XII); 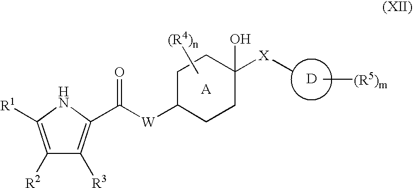
and thereafter if necessary:
i) converting a compound of the formula (I) into another compound of the formula (I);
ii) removing any protecting groups;
iii) forming a pharmaceutically acceptable salt.

15. A compound of formula (I) or a pharmaceutically acceptable salt thereof as claimed in claim 1 wherein R¹ is methyl.

16. A compound of formula (I) or a pharmaceutically acceptable salt thereof as claimed in claim 1 wherein R² is chloro.

17. A compound of formula (I) or a pharmaceutically acceptable salt thereof as claimed in claim 1 wherein R³ is chloro.

18. A compound of formula (I) or a pharmaceutically acceptable salt thereof as claimed in claim 1 wherein R⁴ is a substituent on carbon and is selected from hydroxy or C₁₋₄alkoxy.

19. A compound of formula (I) or a pharmaceutically acceptable salt thereof as claimed in claim 1 wherein R⁵ is a substituent on carbon and is selected from carboxy, N—(C₁₋₄alkoxy)carbamoyl, C₁₋₄alkylS(O)ₐ wherein a is 2, C₁₋₄alkoxycarbonyl, C₁₋₄alkylsulphonylamino or heterocyclyl-R¹¹—; wherein R¹¹ is —C(O)—.

20. A compound of formula (I): 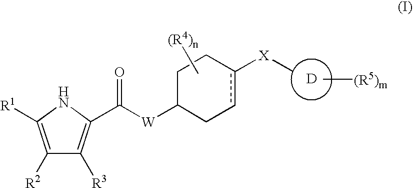
wherein:
R¹ is methyl;
R² is chloro;
R³ is chloro;
W is —NH—;
Ring A is cyclohexyl or cyclohexenyl wherein the dashed bond is a single or a double bond;
X is a direct bond;
Ring D is phenyl;
R⁴ is a substituent on carbon and is selected from hydroxy or methoxy;
R⁵ is a substituent on carbon and is selected from carboxy, N-(methoxy)carbamoyl, mesyl, methoxycarbonyl, ethoxycarbonyl, mesylamino or morpholinocarbonyl;
n is 0 or 1;
m is 1 or 2; wherein the values of R⁵ may be the same or different;
or a pharmaceutically acceptable salt thereof.
